(19) United States
(12) Patent Application Publication
(10) Pub. No.: US 2006/0204969 A1
(43) Pub. Date: Sep. 14, 2006

(54) Genetic variations (SNPs) adjacent to the AKT1 gene locus, and diagnostic and prognostic uses thereof

(76) Inventors: Eric P. Hoffman, Kensington, MD (US); Joseph M. Devaney, Silver Spring, MD (US)

Correspondence Address: DR. MELVIN BLECHER, 4329 VAN NESS ST., NW, WASHINGTON, DC, 20016, US

(21) Appl. No.: 11/035,377

(22) Filed: Jan. 14, 2005

(51) Int. Cl. C12Q 1/68; C07H 21/04

(52) U.S. Cl. 435/6; 536/23.2

(57) ABSTRACT

We have identified and isolated a 12 kb region immediately upstream of the AKT1 gene containing eight single nucleotide polymorphic polynucleotides (SNPs) in 4 haplotype regions that show strong association with body composition, Basal Mass Index, and AKT1 expression enhancement in human males. A four-locus haplotype (Haplotype 2) was defined where residues within highly conserved regulatory regions were altered. This haplotype explained up to 26% of population variation in bone cortical volume, 12% of subcutaneous fat volume, and 9% of strength variation, resulting in a body build with large bones, strong muscles, and low subcutaneous fat. Other SNPs detect, presymptomatically, the potential for increased amounts of subcutaneous fat and Type II diabetes. The detection of these SNPs by the techniques described herein forms the foundation for genotype-specific clinical interventions designed to slow the rapid population increases in obesity and Type II diabetes.

### FIELD OF THE INVENTION

The invention is generally related to defining quantitative trait loci (QTLs) that contribute to body composition. More specifically, the invention relates to SNP sequences derived from a region immediately adjacent upstream to the AKT1 gene that are associated with anthropomorphic variables, volumetric and cross-sectional MRI (muscle, mass and strength, subcutaneous fat, and cortical bone), and the use of these SNPs for both diagnostic and clinical intervention methods.

### BACKGROUND

The incidence of obesity, metabolic syndrome, and type II diabetes is an epidemic in most industrialized populations (1). Obesity, as defined by body mass index (BMI)>30 has risen in the USA from ˜10% of women in 1990, to ˜20% of women in 2002, with three states reporting >25% obesity rates (2). African-American and Hispanic populations are at significantly higher risk, and it has been calculated that a Hispanic child born in 2000 will have about a 50% risk of developing type II diabetes in his or her lifetime, with an associated loss of 18-22 quality-adjusted life years (3).

Type II diabetes is characterized by persistent hyperglycemia in the face of adequate amounts of circulating insulin and a functional pancreas, hence, is also referred to as insulin-independent diabetes. Because it generally occurs in individuals over the age of 40, it also has been referred to as late-onset diabetes. The dramatic rise in obesity in the United States has lead to an equally alarming increase in the percentage of the population who suffer from the metabolic syndrome. “Metabolic syndrome” is a clustering of athersclerotic cardiovascular disease risk factors, such as hypertension, dydlipidemia, insulin resistance, low levels of HDL and a systemic proinflammatory state, impaired fibrinolysis, procoagulation and, most telling, central obesity. Indeed many experts contend that insulin resistance is the primary cause of Type II diabetes and that it is closely correlated to visceral adiposity (obesity). It should be pointed out that obesity alone does not always lead to insulin resistance, and vice versa. Such observations point to the added role of genetics in the acquisition of Type II diabetes.

It should also be borne in mind that muscle cells are a primary insulin target tissue and the primary storage depot for glycogen. Hence, insulin resistance may also be expected to play a role in the utilization of circulating glucose by muscle cells.

There are three primary and inter-related causes of the rapid increase in obesity and metabolic syndrome; inactivity, easy access to inexpensive high caloric food, and genetics (4). Of these three, genetics plays a dominant role, in that there are genetic propensities to become obese and insulin resistant, independent of ethnicity, extent of inactivity and food intake. This genetic predisposition extends through all populations, with body mass index (BMI) as one harbinger of obesity and metabolic syndrome thought to show 75% heretibility (5). The inter-individual variation in energy balance “set point” is thought to be the purposeful balance of risk of starvation (promotion of fat storage), and the risk of predation (strength and leanness) within recent human evolution (6). Despite the acknowledged importance of genetic factors in an individual's set point for energy balance, the specific genetic risk factors are poorly understood. The genetics of energy balance, adiposity, and insulin resistance are undoubtedly complicated, with genetic factors responsible for baseline values (e.g. baseline adiposity or muscle mass), and genetic determinants of response to environment (e.g. energy balance, response to hyperglycemia). Moreover, one cannot isolate a particular organ easily; fat tissue, muscle, liver, pancreas, and brain function are all intimately intertwined via endocrine functions of each.

It would be extremely useful to identify genetic loci that are diagnostic and prognostic for body compositions, muscle size and strength, bone size and adiposity. Such genetic loci have been discovered, and are described below.

### SUMMARY OF THE INVENTION

We have Identified and isolated eight single nucleotide polymorphic (“SNP”) polynucleotides derived from a 12 kb region immediately upstream of the AKT1 gene in human males, seven of which are highly conserved among all races and animal species tested, constituting a group of SNPs consisting of SEQ ID Nos. 1 through 8, or complementary strands of SEQ ID Nos. 1 through 8. These SNPs exhibit strong association with certain aspects of body composition in human males, with these aspects consisting essentially of the states of bone cortical area and volume, subcutaneous fat area and volume, muscle area, volume and strength, and Body Mass Index that is strongly correlated with the potential for Type II diabetes.

The alleles of SEQ ID Nos. 1 through 8 are, respectively, -GI71T, -C8541T, -C12293A, -A8665G, -G738A, -G143A, -C3349G, and -G8371T. [009] In one embodiment, SEQ ID Nos. 1, 2, 3 and 4 are members of a 4-allele haplotye (“Haplotype 2”) that is associated with increased baseline muscle strength, decreased subcutaneous fat, and larger bones, and a potentially decreased potential for Type II diabetes.

In still another embodiment, SEQ ID NO. 5 constitutes Haplotype 4 that is associated with increased muscle cross section area and volume, but with no effect on subcutaneous fat or strength.

In yet another embodiment, SEQ ID NO, 8 constitutes Haplotype 1 that is associated with increased amounts of subcutaneous fat, an increased Body Mass Index, and an increased potential for Type II diabetes.

Additional embodiments constitute methods for analyzing a subject's genomic DNA for the presence of one or more of SEQ ID Nos 1 through 8, or complementary strands thereof, in order to predict presymptomatically the likelihood that a human male will have a genetic propensity for a particular quality involving one or more of bone size, muscle size and strength, amount of subcutaneous fat, the Body Mass Index (“BMI”) and the potential for Type II diabetes.

In another embodiment, the method of the invention is used to determine the presence of the alleles of Haplotype 2 (SEQ ID Nos. 1-4) in order to determine whether the human male will have a genetic propensity for larger bones, stronger muscles, lower subcutaneous fat and BMI, and a decreased potential for Type II diabetes.

In another embodiment, the method of the invention is used to determine the presence of the allele of Haplotype 1 (Seq ID NO. 8) in order to determine whether the subject will have a genetic propensity for excessive subcutaneous fat and obesity, and increased BMI and an increased potential for Type II diabetes.

In another embodiment, the method of the invention is used to determine the presence of the allele of Haplotype 4 (SEQ ID NO. 6) in order to determine whether a human male will have a genetic propensity for increased muscle cross sectional area and volume, no effect on subcutaneous fat or muscle strength, and a decreased potential for Type II diabetes.

In another embodiment, the method of the invention is used to test for the presence of the allele of SEQ ID NO. 1 which, if present, indicates that the human male has a genetic propensity for increased bone cross sectional area and volume and decreased subcutaneous fat.

In still another embodiment of the invention, the method of the invention is used to test for the presence of the allele of SEQ ID NO. 1 or 6, which are protein expression enhancers of the AKT1 gene in muscle.

Another embodiment constitutes detection reagents comprising one or more polynucleotides of the group consisting of SEQ ID No. 1 through 8, or complentary strands thereof, that is suitable for predicting the efficacy of clinical interventions designed to improve body compositions related to muscle strength, subcutaneous fat, bone size, and a potential for avoidance of Type II diabetes.

It is also within the scope of this invention to prepare commercial kits that contain the inventive detection reagents

### FIGURES

FIG. 1. Muscle strength in males is associated with AKT1-C12,273T genotype in all ethnic groups. Males with different AKT1-C12,273T genotypes were seen to show significantly different quantitative strength measurements after adjustment for height, weight, and age. Stratification of data by self-reported ethnic group showed similar associations with the rare allele (T) and increased baseline 1-RM strength.

FIG. 2. Map of the AKT1 locus. Exons of the AKT1 gene are shown as vertical blue lines, with the refseq promoter/first exon (P1), and three alternative promoters/first exons defined by ESTs (Alt P). The 12 polymorphisms identified and discussed in this study are shown, with nucleotide position relative to the transcriptional start site of the refseq transcript (P1). An extended CpG island upstream of AKT1 is indicated by the green box, as well as a potential, uncharacterized transcript unit (ZNF). The region containing the CpG island and potential ZNF transcript is expanded to show the conservation of this region through Fugu and Zebrafish. AKT1 exons are also highly conserved.

FIG. 3. The AKT1 haplotype 2 is associated with increased baseline bone cortical CSA and volume, and decreased subcutaneous fat CSA and volume in males. Shown is gene association data for the -171 locus (haplotype 2), with correlation of semi-automated cross-sectional and volumetric MRI data for 51 males. Homozygotes for haplotype 2 are seen to show larger bones and less subcutaneous fat. When data is stratified for weight into two groups, all results remain statistically significant.

FIG. 4. The AKT1 upstream haplotype defined by -738 is associated with increased muscle cross sectional area in males. 51 males from the FAMuSS cohort were studied by semi-automated MRI analyses (Rapidia), for association with muscle, bone, and subcutaneous fat cross-sectional area, and volume. Genotypes at the -738 locus (Haplotype 4) were found associated with muscle cross-sectional area and volume. The -738 polymorphism is relatively rare, and no homozygous were seen in the 51 subjects studied.

FIG. 5. Body mass index and -C171T genotype. Shown are genotype×BMI associations for the 945 subjects in the FAMuSS cohort. Both males and females show a trend towards lower BMI with increasing dose of haplotype 2 (-171T). This is consistent with the larger bones, stronger muscles, and lower subcutaneous fat associated with haplotype 2 in males (FIGS. 1, 3, 4).

FIG. 6. Conservation of haplotype 2 SNPs, and potential transcription factor binding sites. Note that all sequences are shown relative to the human reference sequence, and are thus inverse complements relative to AKT1. Only human, chimpanzee, mouse, rat, and dog are shown, although all there is also strong conservation through fish (see FIG. 2). The base altered by the polymorphism is shown in green, and completely conserved residues shown by blue highlight. Potential transcription factor binding sites that may be altered by the polymorphism are shown below each region. The name of the DNA binding protein is associated with the polymorphic allele that would retain the binding site. For example, with -G171T, the T allele loses an RREB site, but gains a Pax5 site.

### DETAILED DESCRIPTION OF THE PREFERRED EMBODIMENTS

We have identified and isolated eight single nucleotide polymorphisms (SNPs) in the 12 kb region immediately upstream of the AKT1 gene in human males, seven of which are highly conserved among humans, all races and various animal species, that are closely associated with different muscle, adipose tissue and bone physiotypes, and that are diagnostic and prognostic for body composition and muscle strength. The eight SNPs can be localized to one or more of four haplotypes that are differently predictive of bone cortical area, subcutaneous fat area and volume, muscle area, volume and strength, BMI and by inference insulin resistant Type II diabetes. The availability of these SNPs as novel detection reagents provides genetic detection methods that predict the success or failure of clinical interventions designed to influence body composition and strength and, possibly, also Type II diabetes. These detection reagents can be constituted in commercial kits for laboratory diagnostic purposes.

We have also discovered that two of these SNPs, SEQ ID Nos. 1 and 6, enhance protein expression of the AKT1 gene in muscle myotubules in the presence of a promoter and appropriate factors. This provides yet another physiological function for the inventive SNPs.

The data from the experiments described below demonstrate how the eight SNPs were discovered and isolated, and their diagnostic and prognostic uses.

### EXAMPLES

### Example 1

### The Study Design of the Factors Affecting Muscle Strength and Size (FAMuSS) Program

We have recently reported the design of the study (7). Briefly, 945 volunteers (18-40 yrs; mean 24±6 yrs; Table 1), were enrolled by one of seven exercise physiology and kinesiology sites. Anthropomorphic data was obtained at study entry, and blood taken for genetic studies. Quantified phenotypes included muscle strength by maximum voluntary contraction (MVC) and one repetition maximum (1 RM), and muscle, bone and fat size by magnetic resonance imaging (MRI) (8). Cross-sectional area (CSA) of the biceps was done using analysis of images at the center of the muscle. In addition, position-corrected semi-automated CSA and volumetric measurements of cortical bone, subcutaneous fat, and entire arm muscle were done for a subset of participants (9).

### Example 2

### Survey of SNPs

Fifty (50) single nucleotide polymorphisms (SNPs) in candidate genes were selected for analyses of genotype associations with age-, weight- and height-adjusted quantitative phenotypes. Candidate genes were selected from our mRNA expression profiling studies in human volunteers and rats (10,11,12,13), previous genetic association studies (14) (Table 2), and biochemical pathway information (15). SNP discovery was done for 25 of these genes by denaturing high pressure liquid chromatography (DHPLC) of all exons, exon/intron boundaries, and selected 5′ and 3′ UTR and promoter sequences in 96 ethnically diverse individuals (Supplemental Table 1) (16). Any identified polymorphism that showed an allele frequency of >10% in the screening panel was used for genetic association studies in the FAMuSS cohort (Supplemental Table 2) (17).

QTLs for ciliary neurotrophic factor (CNTF) and muscle strength, angiotensinogen converting enzyme (ACE) and change in muscle strength and baseline body mass, PPARG and body weight, interleukin 6 (IL6) with change in cortical bone cross sectional area following training, and insulin-like growth factor 1 (IGF1) with body weight were validated (Table 2). An alpha-actinin 3 (ACTN3) polymorphism that results in complete loss of the protein has been associated with elite power athletes, and we recently reported an association with muscle baseline strength in females (18). However, we were able to validate the previous findings for only men or women, and only rarely both (Table 2). In most instances, the previously identified QTL explained only a relatively small amount of the total variation seen, the exception being the IL6 polymorphism and bone remodeling, where 12.6% of variation in training-induced bone remodeling could be attributed to this polymorphism, similar to the dramatic effects of this polymorphism seen in Danish military recruits (Table 2).

### Example 3

### Another Survey of Candidate Gene SNPs

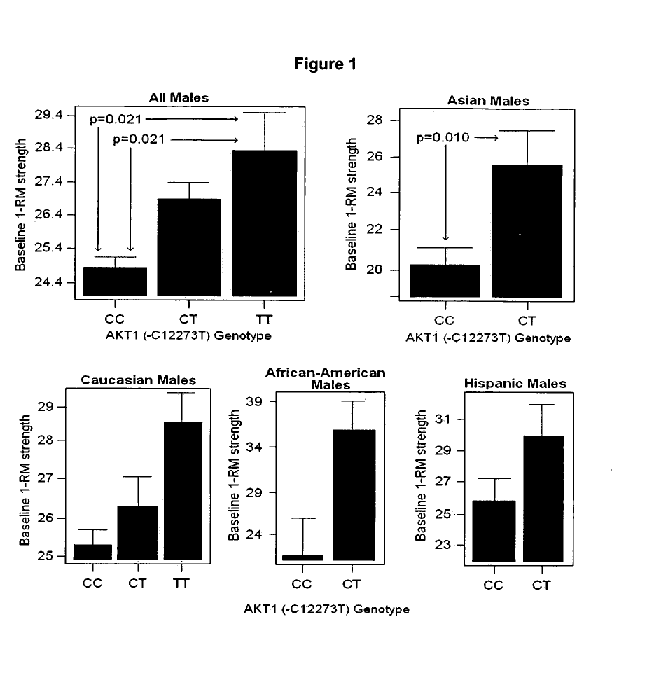

Forty four (44) candidate gene SNPs drawn from emerging biochemical pathway data on muscle atrophy and hypertrophy, as well as genes that we have found strongly regulated by aerobic or resistance activity of muscle were tested (Supplemental Table 2). The most significant associations in our study were initially seen with a novel SNP near the AKT1 gene with baseline muscle strength in males (Table 2). AKT1 was considered a strong functional candidate, due to its central role as a signaling molecule in the regulation of muscle remodeling during both atrophic and hypertrophic stimuli (19,20). The SNP was mapped within the 5′ UTR of the AKT1 gene in the 2002 construct of the human genome; however, the 2003 updates of the genome sequence data placed this SNP 12 kb upstream of the first exon. The -C12,273A AKT1 polymorphism showed a relatively high allele frequency in all populations tested (allele: Caucasians 29%; Asians 15%; African-Americans 34%; Hispanics 23%) (Supplemental Table 4). Male subjects showed a quantitative effect of genotype, with the strength of CC<CT<TT (FIG. 1), with genotype explaining about 9% of all variation in baseline strength in males (Table 2).

### Example 4

### Association Determinations

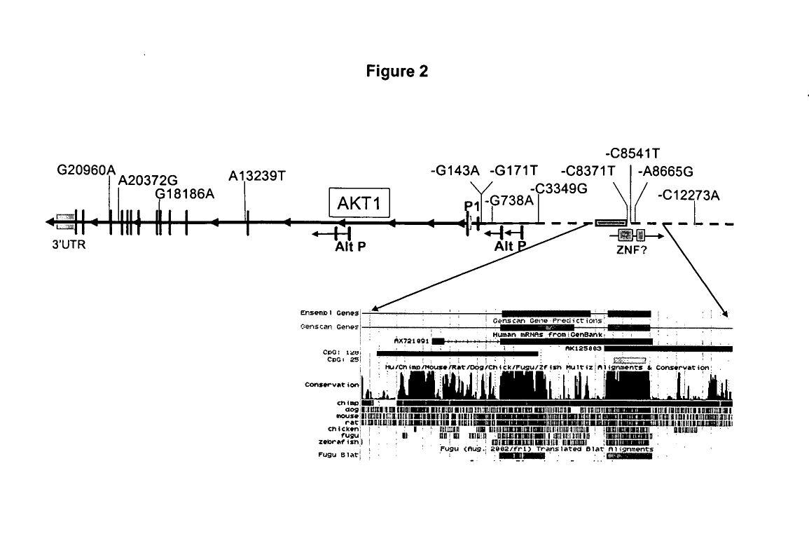

To further interrogate the association with AKT1 polymorphisms with body composition, we conducted a thorough SNP discovery of the AKT1 gene and upstream promoter region sequence. We identified 12 polymorphisms in and upstream of AKT1 covering a 35 kb region (FIG. 2). To define haplotypes across the AKT1 locus, we genotyped the 945 subject FAMuSS cohort for all 12 SNPs, as well as the 96 individual screening panel, did pairwise testing for linkage disequilibrium in both the entire cohort, and then stratified by ethnic subgroup (21) (Supplemental Tables 4, 5). This analysis showed that the eight SNPs upstream of AKTI (-143 to -12,273) formed four relatively common haplotypes (FIG. 2; Table 3). While the ancestral haplotype (haplotype 1) was the most common in all ethnic groups tested, we found three additional haplotypes showing multiple loci in linkage disequilibrium (Table 3). Haplotype 2 was comprised of 4 loci, and haplotype 3 of two distinct loci; both were found in all ethnic groups tested, suggesting that they may have pre-dated ethnic migration. Haplotype 3 was much more common in persons of Asian decent, and haplotype 4 involved a single locus at -738 that superimposed on other haplotypes (Table 3). Polymorphisms within the AKT1 transcript unit itself formed distinct haplotypes that were not in linkage disequilibrium with any of the upstream polymorphisms.

### Example 5

### Further Testing of Associations

We re-tested the FAMuSS muscle strength and size variables against seven of the 12 AKT1 polymorphisms, and found that both the -171 and -12,273 rare alleles showed association with baseline strength in males, consistent with their placement in the same haplotype (Supplemental Table 1; Table 3). The -738 locus showed association with baseline biceps cross sectional area by MRI, with the rare allele showing a quantitative increase in size (GG<GT<TT) (Supplemental Table 1; Table 2). No significant associations were found with any of the four polymorphisms within the AKT1 transcript unit itself.

### Example 6

### MRI Analyses and Haplotype Discovery

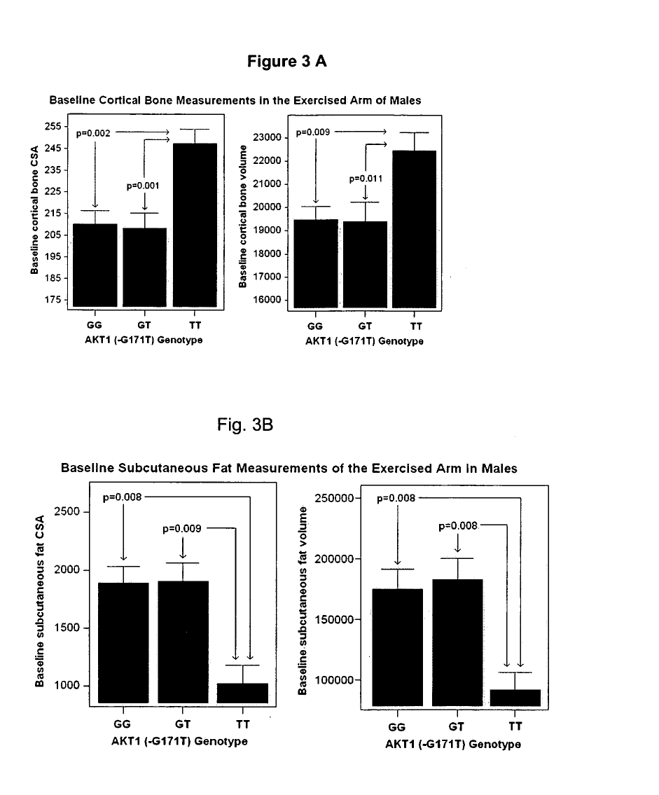

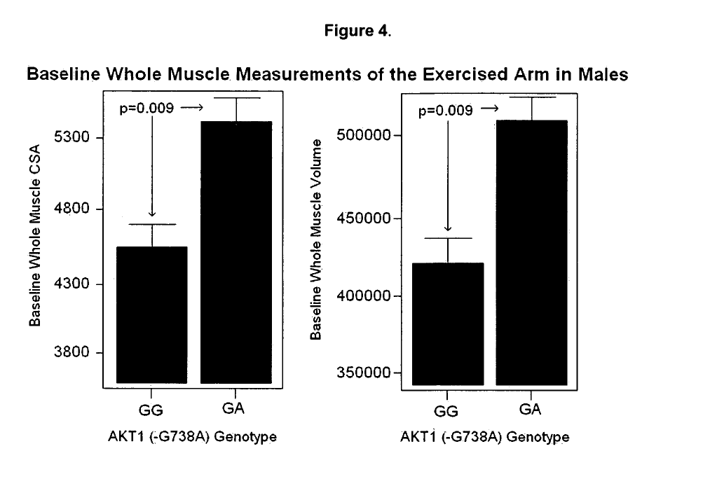

To extend our studies of the AKT1 locus, 51 males were selected for semi-automated cross-sectional and volumetric MRI analyses of whole arm muscle, subcutaneous fat, and cortical bone (Supplemental Table 6). This analysis showed that the two distinct haplotypes upstream of the AKT1 gene were associated with different phenotypes. The haplotype marked by the -171T allele showed strong association with increased bone cortical cross-sectional area and volume, and decreased subcutaneous fat in males (FIG. 3) (Table 2), while the haplotype defined by -738 A (haplotype 4) showed association with increased baseline muscle cross-sectional area and volume in males (FIG. 4; Table 2), consistent with the Matlab cross sectional area in the entire cohort (Supplemental Table 1). This data shows that males that are homozygous for -171T haplotype are stronger, show larger bones, and less subcutaneous fat than other haplotypes. On the other hand, males homozygous for haplotype 4 show larger muscle volume, with no effect on fat, bone, or strength. There was no association with any AKT1 polymorphism and traits in females.

### Example 7

### Variability Due To Genotype Effects

We studied the variability due to genotype effect for both haplotypes in males, and found that about 25% of all variation in cortical bone volume could be explained by the -171T haplotype, as well as 12% of variation in subcutaneous fat volume and 9% of muscle strength. The -738T genotype explains about 10% of variation in muscle size (volume) in males. The variability associated with AKT1 polymorphisms is considerably larger than other QTLs for muscle strength and body composition identified to date (Table 2).

### Example 8

### Body Mass Indeces

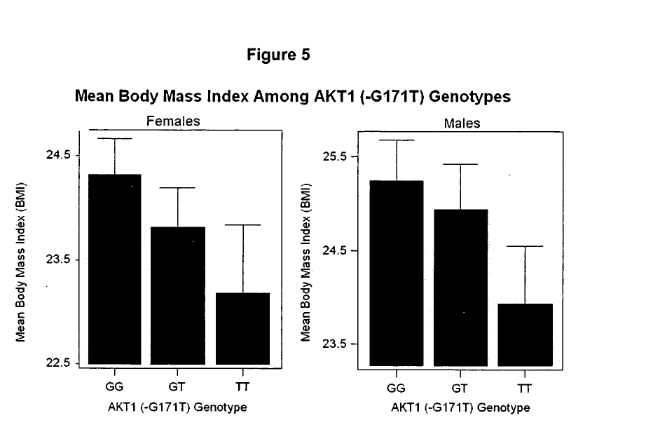

Body mass index (BMI) is often used as a measure of body type and fat content. BMI is based on height and weight only, without any quantitative measures of the relative contribution of muscle, fat and bone to the overall weight. We tested the -G171T locus against BMI in the 945 subject cohort, and found that BMI tracked with genotype in males, as expected, with BMI GG>GT>TT (FIG. 5). Importantly, the same locus also tracked with BMI in women, with similar quantitative effect of genotype as males (FIG. 5). Again, associations were not statistically significant, likely as a result of the large variance in BMI measures.

The present data demonstrate that haplotypes upstream of the AKT1 gene are a major determinant of body composition and muscle strength in males. AKT1 (also called PKB) has emerged as a key signaling molecule, with many membrane-associated and intracellular signaling pathways converging on AKT1, and AKT1 then controlling diverse cellular growth and cell response pathways through phosphorylation of other proteins (15). AKT1 is an important factor for phosphatidylinositol 3-kinase signaling initiated by numerous growth factors and hormones, including those involved in protein synthesis and controlling rates of gene transcription (24). AKT also phosphorylates several proteins involved in cell development and death pathways, providing a negative regulator of apoptosis (10).

### Example 9

### Consequences of Alleles on AKT1 Structure and Function

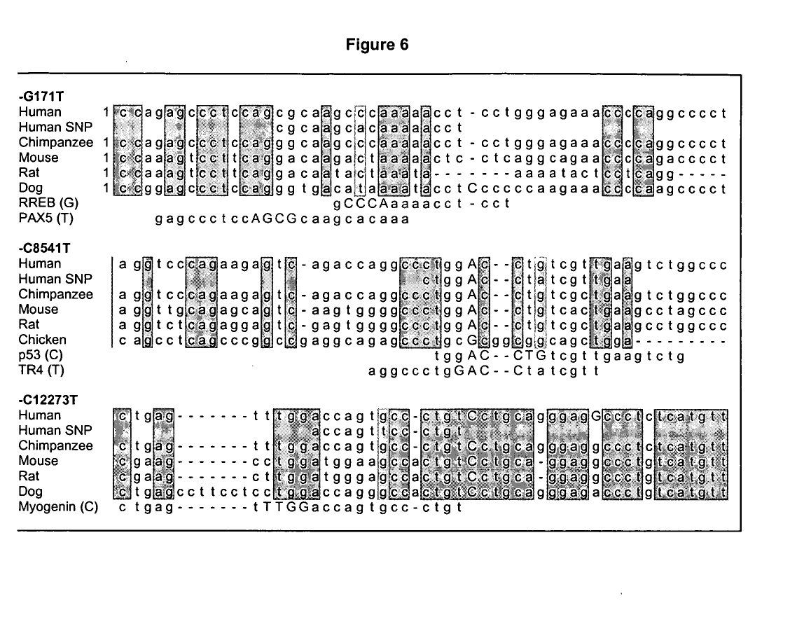

We defined the possible consequences of the base changes on AKT1 structure or function. We first tested each of the 12 polymorphisms for conservation through evolution. We found that four of the 12 polymorphisms were in blocks of high conservation (-C12273A; -A8665G; -C8541T; -G171T), and three of these were in the same Haplotype 2 (FIG. 6). Particularly large blocks of sequence conserved through fish (zebrafish, Fugu) were seen about 6 kb upstream of the AKT1 refseq start site (FIG. 2). AKT1 also shows three additional promoters defined by EST sequences, with the most 5′ about 3 kb upstream of the refseq promoter, and hence 3 kb from the strongly conserved regions (FIG. 2). There is also a strong CpG island, and a potential yet poorly defined transcript unit that may include some of these regions of conservation, with a predicted protein of 100 amino acids containing transcription factor motifs (ZNF, FIG. 2) (26). Thus, the highly conserved regions (FIG. 2, FIG. 6) could alter transcription of AKT1, and/or transcription of coding sequence of the hypothetical transcript upstream of AKT1.

A Haplotype 2 was defined as a QTL for muscle, bone and fat phenotypes in our cohort, we further studied the three conserved loci that comprise this haplotype (-171, -8541, -12,273) (FIG. 6). The polymorphisms at these three loci each changed the consensus binding sites for transcription factors (27). We found that previously characterized effects of perturbations of each of these transcription factor binding sites literature associations with muscle, bone, and fat cells are intriguing, and suggest possible coordinate regulation by these three conserved sequences to orchestrate a balance of these tissues. Specifically, the -171T allele adds a potential PAX5 binding site (28). Pax5 knockout mice show an early osteopenic phenotype, with increased activity of osteoclasts (29). Normal induction of Pax5 during osteogenesis would lead to an increased production of AKT1 in haplotype 2, but not haplotype 1. Thus, the pro-osteogenic Pax5 activity could be potentiated by co-induction fo AKT1, leading to the increased bone size we found associated with haplotype 2. Pax5 is not thought to be important for muscle or bone.

The -8541 allele removes a potential p53 binding site, while adding a nuclear hormone receptor TR4 binding site (30, 31). TR4, like Pax5, is highly expressed in bone, and may positively regulate bone development and homeostasis, with potentiation by increased AKT1 expression in Haplotype 2 (32). With regards to p53, pre-adipocytes are protected from apoptosis by Wnt signaling via AKT1 (33), and induction of AKT1 in pre-adipocytes would be expected to increase the amount of fat cells. Importantly, p53 has recently been shown to be part of a negative feedback loop that senses the fed state, and down-regulates lipogenesis in adipocytes (34). Thus, we hypothesize that the normal induction of p53 in adipocytes by a meal in obese subjects results in the increased expression of AKT1 in subjects with haplotype 1, leading to increased numbers and/or sizes of fat cells. Again, this is what we observed in our association studies, with haplotype 1 associated with increased subcutaneous fat and BMI, and haplotype 2 with decreased fat and BMI. p53 has also been found to be important in bone remodeling. Normally, limb immobilization leads to concomitant loss of muscle and bone. Muscle loss is dependent on the AKT1/Foxo/atrogin1 ubiquitin ligase pathway (35), while bone loss appears dependent on p53 (36). Mice deficient in p53 no longer show any loss in bone following limb immobilization.

The -C12,273A allele removes a potential myogenin/nuclear factor 1 binding site (36); myogenin is a key co-activator of MyoD, and is responsible for driving muscle differentiation (FIG. 6). AKT1 typically prevents apoptosis and thereby promotes cell proliferation in most cells types. We would expect that the induction of AKT1 by myogenin during muscle development or regeneration in haplotype 1 may counteract the pro-differentiation myogenin signal. The effects of this are difficult to predict. Moreover, each of these three polymorphism may act upon the upstream transcript unit (Znf?), and the function of the putative encoded protein are wholly unknown. Preliminary data suggests that the transcript is not expressed in mature human muscle.

AKT1 has been shown to be important for muscle, bone, and fat tissues, both in development and homeostasis. The phenotype AKT1 mice is primarily that of growth retardation and increased apoptosis (38), but AKT2 is expressed in most tissues and is thought to be partially functionally redundant with AKT1 (39). AKT2 null mice are insulin resistant, but double knockouts for AKT1/AKT2 show abnormalities of muscle, bone development, and impeded adipogenesis (39). These are the same tissues that we have shown to be influenced by QTLs upstream of AKT1 in the human genetic association data reported in this current study. AKT1 is also increasingly recognized for its role in metabolism and insulin signaling. Although AKT2 is more highly expressed in insulin-sensitive tissues, both AKT1 and AKT2 are downstream of the key phosphatidylinositol 3-kinase pathway (PI3k). The P13k pathway is critical for a cell's response to leptin, regulation of the insulin receptor, response to IGF-1, and many other signaling pathways (40,41). AKTI is involved in intramuscular insulin signaling and has also been linked to muscle hypertrophy and angiogenic growth factor synthesis (42,43,44).

The easy availability of relatively inexpensive, high calorie food, coupled with low physical activity levels are driving the rapid rise in obesity and type II diabetes. Indeed, poor diet and low physical activity are expected to overcome tobacco as the single most common cause of premature death (45,46). However, it is also clear that individuals show different propensities to become obese, with certain genetically derived “physiotypes” that help set lean body mass (muscle content), bone size, and fat deposits (47,48). An individual's genetically-determined tendency to become obese or remain lean seems conserved throughout primates. For example, in carefully controlled studies of rhesus monkey populations provided unlimited food only a subset of individuals become morbidly obese (49). These same studies have carefully studied the progression from obesity to type 11 diabetes, and have shown that muscle insulin resistance is one of the earlier stages of this process. Thus, fat tissue and muscle show endocrine actions that work together with the pancreatic beta cells and liver to regulate energy balance throughout the body, and each of these tissues contribute to a specific energy balance “set point” specific to each individual (genetically determined), yet influenced by environment. The identification of genetic risk factors for obesity-related physiotypes could be considered a key first step in developing personalized interventions to prevent obesity and the associated morbidity factors.

The present data demonstrates that transcriptional regulation of AKT1 is a major genetic determinant of physiotype in males. The four locus haplotype 2 is associated with larger bones, stronger muscles, and lower subcutaneous fat. The high degree of conservation of three of the four loci, and the establishment of this haplotype prior to ethnic divergence, suggests that there may be coordinate regulation of multiple promoter elements. This would allow a balancing of the muscle, fat, and bone in an environmentally-sensitive manner, leading to physiotypes adapted to distinct environmental conditions. The present data also suggests that the ancestral haplotype (Haplotype 1) results in a physiotype better equipped to survive food deprivation, while the second common haplotype in all world populations (Haplotype 2) imparts a larger boned and stronger build better able to ward off predators. This genetically determined physiotype is highly significant for males but not females, suggesting that the genetic loci responsible for physiotypes in the two sexes are likely different. Given the strong predictive correlations between BMI, subcutaneous fat, and risk for subsequent obesity, metabolic syndrome and type II diabetes, our data suggests that genetic testing for Haplotype 2 will identify individuals at lower risk for poor muscle strength, weak bones, obesity, metabolic syndrome and type II diabetes.

### Example 10

### The Role of SEQ ID Nos. 1 and 6 in Enhancing Protein Expression of the AKT1 Gene in Muscle.

SEQ ID Nos. 1 and 6 SNPs were attached to pGL3 basic and promoter vectors and luciferase expression was examined in myoblasts and myotubules. Attachment to the basic vector actually inhibited expression both myoblasts and myotubules. However, attachment to the promoter vector did not change expression in myoblasts, but significantly activated expression in myotubules. Furthermore, -143AA activated expression more strongly than did -143GG (p<0.008-143GG vs p53L promoter vector only; p<0.005-143AA vs p53L promoter vector only; p<0.027-143AA vs-143GG). The same results were obtained with the -171 SNP. We conclude that these SNPs are expression enhancers in the presence of a promoter and appropriate factors, and that myotubules, but not myoblasts, are active in this regard.

### Example 12

### Method of Use of the SNPs

In the method of the invention, a patient's genomic DNA can be extracted from blood or buccal cells using the PUREGENE DNA Purification System (Gentra Systems, Minneapolis, Minn.) according to the manufacturer's instructions. Briefly, for whole blood the extraction process is as follows: (1) Red blood cells are lysed and the contents of the red blood cells removed; (2) nucleated cells are lysed, thus exposing proteins and DNA; (3) proteins are precipitated and removed; (4) DNA is isolated by alcohol precipitation and placed in a DNA hydration solution. DNA is extracted from nucleated cells as follows: (1) cells are lysed, thereby liberating DNA and proteins; (2) proteins are precipitated and removed; and, (3) DNA is isolated using alcohol precipitation, and placed in a DNA hydration solution.

This DNA is analyzed for the presence of one or more of the 8 allelic nucleotides of SEQ ID Nos. 1 through 8, or their complementary strands by standard methods well known in this art. The pattern of alleles and haplotypes will thereby predict which clinical intervention is best suited for the patient in order to increase muscle strength and bone size and to decrease subcutaneous fat and thus the risk of Type II diabetes.

### REFERENCES AND METHODOLOGIES

To the extent that the following references disclose methods used herein, they are incorporated by reference.
1. A H Mokdad et al. JAMA 289:76 (2003)
2. www.cdc.gov/nccdphp/dnpa/obesity/trend/prev_reg.htm
3. K M Narayan et al. JAMA 290:1884 (2003)
4. J R Speakman, J. Nutr. 134:2090S (2004)
5. L. Perusse et al. Obes. Res. 8:26 (2000)
6. J G Mercer et al. Biobehav. Rev. 25:101 (2001)
7. P D Thompson et al., Med. Sci. Sports Exer. 36:1132 (2001)
8. Entry muscle strength using both one repetition maximum (1 RM) and maximum voluntary contraction (MVC) were done using standarized equipment and protocols. Entry MRI was done on 15 contiguous axial slices (16 mm) from each arm independently, using the top of the bead in a sagittal scout view to loocate the 8^{th} slice going from the top of the arm toward the elbow. Sdcans of both arms were taken by Fast Spoiled Gradient echo with TE 1.8/TR 200 msec (flow artifact suppression). The images had a 22 cm×22 cm field of view with a pixel matrix of 256×256. Subjects then participated in a 12 week supervised resistance training program with the non-dominant arm, and strength and MRI measurements repeated upon exit from the study.
9. We used the Rapidia software (INFITT Technology, www.infinitt.com). For accurate measurements of muscle, bone and subsutaneous fat size customized measurement tools were implemented in Rapidia to obtain cross-sectional volume (6 slices proximal to the metaphysical flare), arm circumference, bone principal moments of inertia, and polar moment of inertia. Semi-automatic segmentation tools in Rapidia were used to separate skeletal muscle structures from overlying fat. Threshold- and tolerance-based region growing techniques were then used with automatic propagation were coded for semi-automatic volumetric measurements. Measurement values were automatically written and saved in a SOL database together with anthropomorphic, strength and genotyping data.
10. Y T Chen, et al. Eur. J. Physiol. 545:27 (2002).
11. D S Hittel et al. J Physiol. 548:401 (2003).
12. Y W Chen et al. J. AppI. Physiol. 95: 2485 (2002).
13. D S Hittel et al. J. Appl. Physiol. In press.
14. T Rankinen et al., Med. Sci. Sports Exerc. 36: 1451 (2004).
15. E P Hoffman et al., Nature (Medicine) 10:584 (2004).
16. SNP discovery was done using a 96 sample screening panel containing genomic DNA from African-Americans, Causanians, Asians and Hispanic subjects. 2′ and 5′ UTR were targeted in each candidate gene, and selected promoter regions (5 kb upstream of the transcriptional start site as defined by refseq sequences (www.genome.ucsc.edu). Overlapping 600 bp regions were amplified by PCR, and tested for heteroduplexes using DHPLC (Transgenomic Wave). Those segments showing similar heteroduplexes in 10 or more individuals in the screening panel were targeted for gene sequencing. Genotyping was done using a novel TaqMan allele discrimination assay that employs the 5′-nuclease activity of Taq polymerase to detect a fluorescent reporter signal generated during PCR reactions. Both alleles were detected simultaneously using allele-specific oligonucleotides labeled with different fluorophores, and genotypes deteremined by the ratio of the 2 fluorophores used. Allele-specific PCR reactions for each SNP included 20 ng genomic DNA, 900 mM forward and reverse PCR primers (Supplemental Table X), 200 nM dluorescent allele discrimination probes (common FAM labeled, rare allele VIC labeled) and TaqMan Universal PCR Master Mix, No AmpErase@UNG (Applied Biosystems, Foster City, Calif.) in a final volume of 20 microliters. PCR and fluorescent ratio [profiles was done using 10 min. at 92° C. and 1 min. at an annealing temperature of 60° C. Reactions were set up using a MWG robot, and fluorescence ratios and allele calling done using an ABI 7700.
17. For genetic association studies, all phenotypes were considered continuous quantitative traits, and were tested by ANCOV A for sidnificance between the 3 genotypes at each focus in sex-stratified data. Ant SNP showing a significant ANCOV A result for a specific quantitative phenotype was then tested in each enthnic subgroup individually. Those SNPs that showed internal variation in multiple ethnic groups, in both sexes, or for multiple phenotypes were retained for further study and evaluation.
18. P M Clarkson et al. J. AppI. Physiol. In press.
19. M Sandri et al. Cell 17:399 (2004).
20. T N Still et al. Mol. Cell 14: 395 (2004).
21. We implemented a “brute force” method to minimize the number of possible haplotypes for a subject population, using each ethnicity separately. Assume that we have L loci to examine for N subjects, and that there are 2 possible genotypes at each locus. That being the case, one can generate 2A(L-1) unique pairs of haplotypes of length L. One constructs a set PH of haplotype pairs as one examines all subjects one by one. For each subject one generates all possible pairs of haplotypes, ph1-phn. If phi is not already in PH. If it already in PH, one increases the hit count for phi in PH. After all subjects are swept on has a set PH where each element has a hit count. The one generates a minimal haplotype H. We add 2 haplotypes to H from a pair in PH in decreasing order of the hit counts, and mark subjects where the 2 haplotypes are present until all subjects are marked. Then one can clearly see which haplotypes are most-commonly shared by most subjects by counting the number of marks for haplotype. This analysis is shown in Supplemental Table 8.
22. D J Glass, Nature New Biology 5:87 (2003).
23 E P Hoffman et al. Nature Med. 10, 584 {2004).
24 H. R. Luo et al. Proc Nail Acad Sci US A. 100′ 11712 {2003).
25 H Cho et al. J Biol Chem. 276:38349(2001)
26 The putative amino acid sequence of the potential transcript contains a BTB domain motif a run of 3 zinc finger C2H2 motifs {NCBI and Expasy). The zinc finger motifs share high amino acid homology to the zinc finger domains found in the zinc finger protein 238 in humans, rats, mice, and the African clawed frog. The zinc finger motifs of the potential transcript (ZNF? In FIG. 2) share 80% identity to the human, rat, and mouse, and 78% with the African tree frog zinc finger motifs found in the ZNF238 protein. The potential transcript is also homologous to many other zinc finger proteins in multiple species only to a lesser extent. The BTB domain {Broad-Complex, Tram track and Bric a brac) is also known as the POZ domain {POxvirus and Zinc finger). It is a homodimerization domain occurring at the N-terminus of proteins containing multiple copies of either zinc fingers of the C2H2 type or Kelch repeats. Many BTB proteins are transcriptional regulators that are thought to act through the control of chromatin structure.
27 Two transcription factor programs were used to define potential transcription factor binding sites; TEss (web site ), and GEMS {Genomatix, Inc.).
28 T Czerny et al. Genes Dev: 2048 {1993).
29 M C Horowitz et al. J Immunol. 173, 6583 {2004).
30 H Aian et al. Oncogene 21: 7901 {2002).
31 A Inga et al. Mol. Cell. Bioi. 22:8612 {2002).
32 H Harada et al. Endocrinology. 139:204 {1998).
33 K Longo et al. J Biol Chem. 277: 38239 {2002).
34 N Yahagi et al. J. Bioi Chem. 278: 25395 {2003).
35 R. Okazaki et al. Ann Rheum Dis. 63: 453 {2004).
36 W D Funk et al. Proc. Natl. Acad. Sci. USA 89:9484 (1992).
38 Z. Y Jiang et al. Proc Nati Acad Sci USA., 100: 7569 (2003).
39 X Peng et al. Genes Develop 17: 1352 (2003).
40 C Duan et al. J. Biol. Chem. 279: 43684 (2004).
41 L H Pearl et al. Proteins 12:761 (2002).
42 J T Brozinick Jr, et al. J Biol Chem. 273:14679 (1998).
43 E Luciano et al. EurJ Endocrinol. 147:149 (2002).
44 A Takahashi et al. Mol Cell Bioi. 22:4803 (2002).
45 L D Caterson et al. Circulation 110:476 (2004).
46 A H Mokdad et al. JAMA. 291:1238 (2004).
47 J R Speakman. J Nutr. 134: 2090S (2004).
48 E E Snyder et al. Obesity Res. 12:369 (2004).
49 N L Bodkin et al. Biol Med Sci. 58:212 (2003).
50 S Roth et al. J. Appl. Physiol. 90:1205 (2001).
51 J Foland,et al. Exp. Physiol. 85:1998 (2003).
52 H E Montgomery et al. Lancet 353:541 (1999).
53 V Lindl et al. Diabetes 51:2581 (2002).
54 S S Dhamrait et al. Eur. J. Appl. Physiol. 89:21 (2003).,
55 G Sun et al. J. Obes. Relat. Metab. Disord. 23,:29 (1999).

56 N Yang et al. Am J Hum Genet 73: 627 (2003).

TABLE 1
Demographics of subject population studied.

| Characteristics | Number (%) |
|---|---|
| Total recruited | 945 |
| Dropouts | 173 (18.3%) |
| Gender * | |
| Female | 530 (58.7%) |
| Male | 305 (41.3%) |
| Ethnicity * | |
| African American | 40 (4.4%) |
| Asian | 72 (7.9%) |
| Caucasian | 710 (78.4%) |
| Hispanic | 47 (5.2%) |
| Other | 35 (3.9%) |

TABLE 2
Summary of positive genetic associations.

| | | Published Findings | | | FAMuSS Findings | | | |
|---|---|---|---|---|---|---|---|---|
| Gene | SNP | N | Phenotype | P | Ref. | Phenotype | N | P value % variation |
| CNTF | -G6A | 494 | Knee extensor | <0.05 | (1) | Baseline isometric | 340 females | 0.004 |
| | | | and flexor strength | | | strength | | 2.7% |
| | | | | | | Change in 1-RM | 346 females | 0.013 |
| | | | | | | strength | | 1.6% |
| ACE | I/D | 33 | Change in muscle | <0.005 | (2) | % change in | 191 | 0.046 |
| | | | strength | | | isometric strength | Caucasian | 3.2% |
| | | | | | | | males | |
| | | 81 | Body weight | 0.001 | (3) | Baseline body mass | 670 males and | 0.06 |
| | | males | Fat mass | 0.04 | | index | females | 0.8% |
| | | | Fat free mass | 0.01 | | | | |
| PPARg | P12A | 490 | Body weight | 0.04 | (4) | Baseline body mass | 320 females | 0.08 |
| | | | | | | index | | 1.3% |
| IL6 | -G572C | 130 | Change in cortical | 0.007 | (5) | Change in bone + marrow | 16 males | 0.008 |
| | | males | bone CSA | | | CSA | | 12.6% |
| | | | | | | (untrained arm) | | |
| IGF1 | -C1245T | 502 | Fat free mass | 0.005 | (6) | Body weight | 603 males and | 0.08 |
| | | | | | | | females | 0.8% |
| ACTN3 | R577X | 429 | Power athletes | 0.01 | (7) | Change in strength | 352 females | 0.05 |
| | | | | | | (Errorl Bookmark | | 2% |
| | | | | | | not defined.) | | |
| AKT1 | Haplotype: | | | | | Baseline strength | 305 males | 0.003 |
| | -C12273T | | | | | | | 9% |
| | -C8541I | | | | | Baseline | 51 males | 0.005 |
| | -G171I | | | | | subcutaneous fat | | 12% |
| | -G143A | | | | | volume | | |
| | | | | | | Post-exercise | 51 males | <0.001 |
| | | | | | | cortical | | 26% |
| | | | | | | bone + marrow | | |
| | | | | | | volume | | |
| | -G738A | | | | | Baseline muscle | 51 males | 0.009 |
| | | | | | | volume | | 10% |

TABLE 3
Haplotypes and populations.

| | | | | | | | | | | | African- |
|---|---|---|---|---|---|---|---|---|---|---|---|
| | -G143A | -G171T | -G738A | -C3349G | -C8371T | -C8541T | -A8665G | -C12273T | Caucasian | Asian | American |
| Hap 1 | G | G | G | C | C | C | A | C | 71 | 35 | 63 |
| ancestral | | | | | | | | | | | |
| Hap 2 | A | T | | | | T | | A | 29 | 15 | 27 |
| Hap 3 | | | | G | | | G | | 10 | 56 | 20 |
| Hap 4 | | | A | | | | | | 5 | 5 | 5 |

SUPPLEMENTAL TABLE 1
SNP discovery for novel genes in 96 ethnically diverse individuals.

| | | | |
|---|---|---|---|
| IGF2 | 2 | 0 | 0 |
| DTR | 6 | 6 | 0 |
| MYF4 (MYOG) | 0 | 0 | 0 |
| MYF6 | 2 | 2 | 0 |
| NNMT | 3 | 1 | 0 |
| IGF1 | 1 | 0 | 0 |
| UCP2 | 9 | 5 | 3 |
| CARP | 13 | 5 | 3 |
| DNAJB1 | 3 | 1 | 0 |
| AKT1 | 47 | 13 | 1 |
| NR4A3 | 2 | 1 | 0 |
| HSPA2 | 1 | 1 | 0 |
| HSPA1A | 2 | 2 | 0 |
| CD44 | 2 | 1 | 0 |
| MLCK | 51 | 26 | 19 |
| GOT1 | 3 | 0 | 0 |
| RESISTIN | 5 | 1 | 0 |
| TNF ALPHA | 3 | 2 | 0 |
| SYNGR2 | 5 | 1 | 0 |
| ANKRD2 | 4 | 1 | 0 |
| MUSCLIN | 3 | 1 | 1 |
| IGFBP7 | 2 | 0 | 0 |
| RNF28 (MURF1) | 4 | 1 | 0 |
| Totals | 173 | 71 | 27 |

SUPPLEMENTAL TABLE 2
Single nucleotide polymorphisms tested for associations
in FAMuSS.

| | | Analyzed in | | | | Analyzed in | |
|---|---|---|---|---|---|---|---|
| | | 166 subject | Analyzed | | | 166 subject | Analyzed |
| | | outlier | in entire | | | outlier | in entire |
| Gene | SNP | population | cohort | Gene | SNP | population | cohort |
| ACE | | X | X | GDF8 | K153R | X | X |
| CNTF | | X | X | GDF8 | A55T | X | |
| ACTN3 | | X | X | GDF8 | 1225T | X | |
| UCP2 | | X | X | GDF8 | P198A | X | |
| GS | S287N | X | X | GDF8 | E164K | X | |
| SYNGR2 | C886T | X | X | TNF | G308A | X | X |
| | | | | alpha | | | |
| APOE | C472T | X | | PPAR | P12A | X | X |
| | | | | gamma | | | |
| NNMT | G5082T | X | X | Resistin | -C180G | X | X |
| PAI-1 | 4G/5G | X | X | Resistin | C30T | | X |
| PGC-1 | G76039A | X | | Resistin | C398T | | X |
| IL6 | -G572C | X | | Resistin | G540A | | X |
| IL6 | -C174G | X | | Resistin | C980G | | X |
| CARP | -C105T | X | X | AKT1 | -C12273T | X | X |
| CARP | A8470G | X | X | AKT1 | -C8678T | | X |
| CARP | Exon 3 | X | | AKT1 | -C8541T | | X |
| | SNP 2 | | | | | | |
| CARP | Exon 3 | X | | AKT1 | -C8371T | | X |
| | SNP 3 | | | | | | |
| CARP | Exon 8 | X | | AKT1 | -C3349G | | X |
| CARP | Exon 5 | X | | AKT1 | -C738T | | X |
| | SNP 1 | | | | | | |
| CARP | Eson 5 | X | | AKT1 | -G171T | | X |
| | SNP 2 | | | | | | |
| CARP | 3UTR | X | | AKT1 | -G143A | | X |
| MLCK | C37885A | X | | AKT1 | A13239T | | X |
| MLCK | G91689T | X | | AKT1 | G18186A | | X |
| MLCK | C49T | X | X | AKT1 | A20372G | | X |
| IGF1 | -T1245C | X | X | AKT1 | G20980A | | X |

SUPPLEMENTAL TABLE 3

| | AKT1 | AKT1 | AKT1 | AKT1 | AKT1 | AK [symbol] |
|---|---|---|---|---|---|---|
| Measure | (-C12, 273A) | (-G738T) | (-G171T) | (A13, 239T) | (G18, 186A) | (A20, 3 [symbol] |
| Baseline | 0.059^{A} | All males (0.003) | NS | NS | NS | NS |
| biceps | | (AA: N = 2: | | | | |
| | | 30.38 ± 3.65) a | | | | |
| | | (GA: N = 18: | | | | |
| | | 24.31 ± 1.22) b | | | | |
| | | (GG: N = 207: | | | | |
| | | 21.12 ± 0.36) a, b | | | | |
| | Asian (0.007) | Other (0.032) | NS | NS | NS | NS |
| | (CC: N = 22: 18.57 ± 0.74) a | (AA: N = 1: | | | | |
| | (CT: N = 9: 22.57 ± 1.16) a | 28.14 ± 4.24) | | | | |
| | (TT: N = 0) | (GA: N = 3: | | | | |
| | | 26.99 ± 1.91) a | | | | |
| | | (GG: N = 8: | | | | |
| | | 18.99 ± 1.24) a | | | | |
| Difference | NS | NS | NS | NS | NS | NS |
| in | Hispanic (0.037) | NS | Hispanic (0.037) | NS | Asian | NS |
| biceps | (CC: N = 6: 3.79 ± .099) a | | (GG: N = 6: 3.79 ± 0.99) a | | (0.004) | |
| | (CT: N = 4: 8.38 ± 1.25) a | | (GT: N = 4: 8.38 ± 1.25) a | | (AA: N = 1: | |
| | (TT: N = 0) | | (TT: N = 0) | | 8.72 ± 1.40) | |
| | | | | | a, b | |
| | | | | | (GA: N = 4: | |
| | | | | | 4.42 ± 0.68) a | |
| | | | | | (GG: N = 28: | |
| | | | | | 3.59 ± 0.27) b | |
| Change | NS | NS | NS | NS | NS | NS |
| in | NS | NS | Caucasian (0.048) | Hispanic | NS | NS |
| biceps | | | (GG: N = 85: 22.22 ± 1.08) a | (0.049) | | |
| | | | (GT: N = 64: 18.18 ± 1.24) a | (No | | |
| | | | (TT: N = 21: 19.28 ± 2.17) | significant | | |
| | | | | differences) | | |
| Baseline | All males (0.003) | NS | All males (0.005) | NS | NS | NS |
| 1- | (CC: N = 133: 24.63 ± 0.52) a, b | | (GG: N = 128: 25.09 ± 0.53) a | | | |
| RM | (CT: N = 91: 28.82 ± 0.62) a | | (GT: N = 90: 25.66 ± 0.63) b | | | |
| | (TT: N = 22: 28.34 ± 1.27) b | | (TT: N = 28: 29.19 ± 1.12) a, b | | | |
| | Asian (0.010) | NS | Caucasian (0.031) | Hispanic | NS | NS |
| | (CC: N = 23: 20.14 ± 1.08) a | | (GG: N = 88: 25.72 ± 0.64) | (0.048) | | |
| | (CT: N = 9: 25.75 ± 1.72) a | | (GT: N = 71: 25.41 ± 0.71) a | (No | | |
| | (TT: N = 0) | | (TT: N = 24: 29.01 ± 1.21) a | significant | | |
| | | | | differences) | | |
| Difference | NS | NS | NS | NS | NS | NS |
| in 1- | NS | NS | NS | NS | NS | NS |
| RM | | | | | | |
| Change | All males (0.026) | NS | NS | NS | NS | NS |
| in | (CC: N = 131: 43.54 ± 1.78) a | | | | | |
| 1-RM | (CT: N = 90: 36.81 ± 2.14) a | | | | | |
| | (TT: N = 0) | | | | | |
| | NS | NS | NS | NS | NS | NS |
| Baseline | NS | NS | NS | NS | NS | NS |
| isometric | NS | NS | NS | NS | NS | NS |
| Difference | NS | NS | NS | NS | NS | NS |
| in | NS | NS | Asian (0.001) | NS | NS | Other (0 [symbol] |
| isometric | | | (GG: N = 25: 14.06 ± 2.94) a | | | (No sign [symbol] |
| | | | (GT: N = 6: 39.25 ± 6.08) a | | | difference |
| | | | (TT: N = 0) | | | |
| Change | NS | NS | NS | NS | NS | NS |
| in | NS | NS | Asian (0.031) | NS | NS | NS |
| isometric | | | (GG: N = 25: 13.46 ± 2.59) a | | | |
| | | | (GT: N = 6: 27.18 ± 5.37) a | | | |
| | | | (TT: N = 0) | | | |

SUPPLEMENTAL TABLE 4
Allele frequencies at the AKT1 locus.

| | | | | African | | | | | |
|---|---|---|---|---|---|---|---|---|---|
| | | Overall | | Americans | | Asians | | Caucasians | |
| | | | Allele | | Allele | | Allele | | Allele |
| AKT1 SNP | Allele | N | Frequency | N | Frequency | N | Frequency | N | Frequency |
| -C12273A | C | 617 | 0.729 | 28 | 0.661 | 56 | 0.848 | 484 | 0.712 |
| | A | | 0.271 | | 0.339 | | 0.152 | | 0.288 |
| -A8665G | A | 611 | 0.829 | 27 | 0.741 | 56 | 0.437 | 480 | 0.891 |
| | G | | 0.171 | | 0.259 | | 0.563 | | 0.109 |
| -C8541T | C | 595 | 0.721 | 25 | 0.600 | 54 | 0.852 | 470 | 0.704 |
| | T | | 0.279 | | 0.400 | | 0.148 | | 0.296 |
| -C8371T | C | 603 | 0.888 | 27 | 1.000 | 56 | 0.964 | 474 | 0.872 |
| | T | | 0.112 | | 0.000 | | 0.036 | | 0.128 |
| -C3349G | C | 606 | 0.875 | 27 | 0.833 | 55 | 0.491 | 476 | 0.927 |
| | G | | 0.125 | | 0.167 | | 0.509 | | 0.073 |
| -G738A | G | 609 | 0.952 | 27 | 0.944 | 56 | 0.938 | 478 | 0.961 |
| | A | | 0.048 | | 0.056 | | 0.062 | | 0.039 |
| -G171T | G | 614 | 0.709 | 28 | 0.625 | 56 | 0.857 | 481 | 0.688 |
| | T | | 0.291 | | 0.375 | | 0.143 | | 0.312 |
| -G143A | G | 607 | 0.856 | 27 | 0.722 | 56 | 0.902 | 476 | 0.851 |
| | A | | 0.144 | | 0.278 | | 0.098 | | 0.149 |
| A13239T | A | 605 | 0.629 | 28 | 0.464 | 55 | 0.491 | 474 | 0.666 |
| | T | | 0.371 | | 0.536 | | 0.509 | | 0.334 |
| G18186A | G | 610 | 0.828 | 28 | 0.839 | 56 | 0.911 | 477 | 0.812 |
| | A | | 0.172 | | 0.161 | | 0.089 | | 0.188 |
| A20372G | A | 613 | 0.556 | 27 | 0.556 | 56 | 0.437 | 481 | 0.573 |
| | G | | 0.444 | | 0.444 | | 0.563 | | 0.427 |
| G20960A | G | 611 | 0.833 | 28 | 0.911 | 56 | 0.723 | 478 | 0.841 |
| | A | | 0.167 | | 0.089 | | 0.277 | | 0.159 |

SUPPLEMENTAL TABLE 5

```
Linkage disequilibrium measurements of the 12 AKT1 loci over
entire cohort, and ethnic subgroups.
Physical map schematic Zinc Finger
              AKT1------------------------<<< [symbol] -----------------
----------→>>>>>>>>>>>>>>>>>>>>>
Species +++ +++ +++ X X X +++
              X X +/− X X
conservation
```

| | | | | | | | |
|---|---|---|---|---|---|---|---|
| AKT1 | AKT1 | AKT1 | AKT1 | AKT1 | AKT1 | AKT1 | AKT1 |
| (-C12273A) | (-A8665G) | (-C8541T) | (-C3349G) | (-G738T) | (-G171T) | (-G143A) | |

Linkage Disequilibrium among AKT1 SNPs: All Ethnic groups

| | | | | | |
|---|---|---|---|---|---|
| AKT1 | | | | | |
| (-A8665G) | | | | | |
| AKT1 | All | | | | |
| (-C8541T) | African Am. | | | | |
| | Asian | | | | |
| | Caucasian | | | | |
| | Hispanic | | | | |
| AKT1 | All | | All | | |
| (-C8371T) | African Am. | | African Am. | | |
| | Asian | | Asian | | |
| | Caucasian | | Caucasian | | |
| | Hispanic | | Hispanic | | |
| AKT1 | | All | | | |
| (-C3349G) | | African Am. | | | |
| | | Asian | | | |
| | | Caucasian | | | |
| | | Hispanic | | | |
| AKT1 | | All | | | |
| (-G738T) | | African Am. | | | |
| | | Asian | | | |
| | | Caucasian | | | |
| | | Hispanic | | | |
| AKT1 | All | | All | All | |
| (-G171T) | African Am. | | African Am. | African Am. | |
| | Asian | | Asian | Asian | |
| | Caucasian | | Caucasian | Caucasian | |
| | Hispanic | | Hispanic | Hispanic | |
| AKT1 | All | | All | | All |
| (-G143A) | African Am. | | African Am. | | African Am. |
| | Asian | | Asian | | Asian |
| | Caucasian | | Caucasian | | Caucasian |
| | Hispanic | | Hispanic | | Hispanic |
| AKT1 | | | | | |
| (A13239T) | | | | | |
| AKT1 | | | | | |
| (G19186A) | | | | | |
| AKT1 | | | | All | |
| (A20372G) | | | | African Am. | |
| | | | | Asian | |
| | | | | Caucasian | |
| | | | | Hispanic | |
| AKT1 | | | | | |
| (G20980A) | | | | | |

| | | | | | | | | |
|---|---|---|---|---|---|---|---|---|
| AKT1 | AKT1 | AKT1 | AKT1 | AKT1 | AKT1 | AKT1 | AKT1 | AKT1 |
| (-C12273A) | (-A8665G) | (-C8541T) | (-C3349G) | (-G738T) | (-G171T) | (-G143A) | (A1) | |

Linkage disequilibrium: complete cohort (n = 995)

| | | | | | | | | | |
|---|---|---|---|---|---|---|---|---|---|
| AKT1 | 1.00 | | | | | | | | |
| (-C8678T) | 0.00 | | | | | | | | |
| | 0.07 | | | | | | | | |
| AKT1 | 0.99 | 1.00 | | | | | | | |
| (-C8541T) | 9265.9 | 0.00 | | | | | | | |
| | 0.95 | 0.08 | | | | | | | |
| AKT1 | 1.00 | 1.00 | 1.00 | | | | | | |
| (-C8371T) | 2000 + | 0.00 | 2000 + | | | | | | |
| | 0.35 | 0.03 | 0.33 | | | | | | |
| AKT1 | 1.00 | 1.00 | 1.00 | 1.00 | | | | | |
| (-C3349G) | 0.00 | 2000 + | 0.00 | 0.00 | | | | | |
| | 0.05 | 0.68 | 0.05 | 0.02 | | | | | |
| AKT1 | 0.16 | 0.39 | 0.08 | 0.07 | 0.71 | | | | |
| (-G738T) | 1.77 | 5.32 | 1.33 | 1.73 | 0.25 | | | | |
| | 0.00 | 0.04 | 0.00 | 0.00 | 0.00 | | | | |
| AKT1 | 0.96 | 0.85 | 0.97 | 1.00 | 1.00 | 0.22 | | | |
| (-G171T) | 881.9 | 0.09 | 1629.5 | 2000 + | 0.00 | 2.08 | | | |
| | 0.83 | 0.06 | 0.87 | 0.31 | 0.06 | 0.01 | | | |
| AKT1 | 0.80 | 0.73 | 0.91 | 0.30 | 1.00 | 0.18 | 1.00 | | |
| (-G143A) | 28.3 | 0.20 | 77.96 | 0.64 | 0.00 | 2.75 | 2000 + | | |
| | 0.29 | 0.02 | 0.37 | 0.00 | 0.02 | 0.01 | 0.41 | | |
| AKT1 | 0.32 | 0.46 | 0.30 | 0.59 | 0.46 | 0.13 | 0.33 | 0.03 | |
| (A13239T) | 3.22 | 4.27 | 2.92 | 6.07 | 3.97 | 1.41 | 3.49 | 0.95 | |
| | 0.07 | 0.07 | 0.06 | 0.08 | 0.05 | 0.00 | 0.08 | 0.00 | |
| AKT1 | 0.00 | 0.54 | 0.02 | 0.42 | 0.46 | 0.80 | 0.12 | 0.01 | 0.60 |
| (G18186A) | 0.00 | 0.36 | 0.97 | 0.50 | 0.45 | 0.16 | 0.81 | 1.09 | 0.25 |
| | 0.00 | 0.01 | 0.00 | 0.00 | 0.01 | 0.01 | 0.00 | 0.00 | 0.04 |
| ATK1 | 0.43 | 0.41 | 0.41 | 0.67 | 0.45 | 0.24 | 0.44 | 0.23 | 0.81 |
| (A20372G) | 3.96 | 3.17 | 3.65 | 6.88 | 3.35 | 1.78 | 4.26 | 1.82 | 38.3 |
| | 0.09 | 0.04 | 0.08 | 0.07 | 0.04 | 0.00 | 0.10 | 0.01 | 0.48 |
| AKT1 | 0.21 | 0.18 | 0.17 | 0.31 | 0.22 | 0.03 | 0.25 | 0.41 | 0.93 |
| (G20890A) | 1.18 | 2.86 | 1.96 | 5.14 | 3.48 | 1.22 | 2.52 | 0.51 | 66.2 |
| | 0.02 | 0.03 | 0.01 | 0.07 | 0.04 | 0.00 | 0.03 | 0.01 | 0.28 |

Linkage Disequilibrium among AKT1 SNPs: African-Americans

| | | | | | | | | | |
|---|---|---|---|---|---|---|---|---|---|
| AKT1 | 0.97 | | | | | | | | |
| (-C8678T) | 0.01 | | | | | | | | |
| | 0.18 | | | | | | | | |
| AKT1 | 1.00 | 1.00 | | | | | | | |
| (-C8541T) | 2000 + | 0.00 | | | | | | | |
| | 0.88 | 0.24 | | | | | | | |
| AKT1 | 1.00 | 1.00 | 1.00 | | | | | | |
| (-C8371T) | 2000 + | 0.00 | 2000 + | | | | | | |
| | 0.03 | 0.01 | 0.02 | | | | | | |
| AKT1 | 1.00 | 0.91 | 1.00 | 1.00 | | | | | |
| (-C3349G) | 0.00 | 109.44 | 0.00 | 0.00 | | | | | |
| | 0.12 | 0.52 | 0.12 | 0.00 | | | | | |
| AKT1 | 0.15 | 0.95 | 0.01 | 1.00 | 0.71 | | | | |
| (-G738T) | 1.59 | 88.24 | 1.03 | 0.00 | 0.23 | | | | |
| | 0.00 | 0.13 | 0.00 | 0.00 | 0.01 | | | | |
| AKT1 | 0.71 | 0.42 | 0.69 | 1.00 | 1.00 | 0.36 | | | |
| (-G171T) | 22.41 | 0.35 | 20.74 | 2000 + | 0.00 | 2.54 | | | |
| | 0.40 | 0.05 | 0.39 | 0.02 | 0.15 | 0.02 | | | |
| AKT1 | 0.89 | 0.94 | 0.89 | 0.63 | 1.00 | 0.29 | 0.93 | | |
| (-G143A) | 110.13 | 0.03 | 74.50 | 7.53 | 0.00 | 2.85 | 102.67 | | |
| | 0.61 | 0.13 | 0.53 | 0.01 | 0.09 | 0.01 | 0.50 | | |
| AKT1 | 0.48 | 0.04 | 0.42 | 0.05 | 0.14 | 0.05 | 0.34 | 0.44 | |
| (A13239T) | 4.60 | 0.89 | 3.87 | 1.12 | 0.79 | 1.12 | 3.16 | 3.70 | |
| | 0.11 | 0.00 | 0.10 | 0.00 | 0.00 | 0.00 | 0.07 | 0.08 | |
| AKT1 | 0.05 | 0.99 | 0.07 | 0.77 | 1.00 | 0.15 | 0.04 | 0.04 | 0.57 |
| (G18186A) | 0.92 | 0.00 | 1.25 | 19.44 | 0.00 | 2.02 | 1.12 | 1.21 | 0.21 |
| | 0.00 | 0.09 | 0.00 | 0.03 | 0.05 | 0.01 | 0.00 | 0.00 | 0.07 |
| ATK1 | 0.10 | 0.54 | 0.07 | 0.20 | 0.46 | 0.65 | 0.20 | 0.11 | 0.45 |
| (A20372G) | 0.77 | 6.23 | 0.82 | 1.57 | 3.83 | 5.65 | 1.06 | 0.77 | 6.32 |
| | 0.00 | 0.15 | 0.00 | 0.00 | 0.07 | 0.03 | 0.00 | 0.00 | 0.18 |
| AKT1 | 1.00 | 0.20 | 0.92 | 1.00 | 0.76 | 1.00 | 1.00 | 0.94 | 0.29 |
| (G20890A) | 0.00 | 2.06 | 0.04 | 0.00 | 28.06 | 0.00 | 0.00 | 0.04 | 0.52 |
| | 0.05 | 0.01 | 0.04 | 0.00 | 0.26 | 0.01 | 0.06 | 0.03 | 0.01 |

Linkage Disequilibrium among AKT1 SNPs: Asians

| | | | | | | | | | |
|---|---|---|---|---|---|---|---|---|---|
| AKT1 | 0.96 | | | | | | | | |
| (-C8678T) | 0.01 | | | | | | | | |
| | 0.21 | | | | | | | | |
| AKT1 | 0.97 | 0.98 | | | | | | | |
| (-C8541T) | 2820.04 | 0.01 | | | | | | | |
| | 0.88 | 0.19 | | | | | | | |
| AKT1 | 1.00 | 1.00 | 1.00 | | | | | | |
| (-C8371T) | 2000 + | 0.00 | 2000 + | | | | | | |
| | 0.21 | 0.05 | 0.22 | | | | | | |
| AKT1 | 1.00 | 0.97 | 1.00 | 1.00 | | | | | |
| (-C3349G) | 0.00 | 572.03 | 0.00 | 0.00 | | | | | |
| | 0.16 | 0.74 | 0.14 | 0.02 | | | | | |
| AKT1 | 0.32 | 0.24 | 0.34 | 0.14 | 0.62 | | | | |
| (-G738T) | 4.74 | 0.56 | 5.49 | 4.07 | 0.21 | | | | |
| | 0.04 | 0.00 | 0.05 | 0.01 | 0.03 | | | | |
| AKT1 | 0.89 | 1.00 | 0.91 | 1.00 | 1.00 | 0.34 | | | |
| (-G171T) | 392.65 | 0.00 | 920.08 | 2000 + | 0.00 | 5.33 | | | |
| | 0.75 | 0.21 | 0.83 | 0.22 | 0.15 | 0.04 | | | |
| AKT1 | 0.94 | 0.88 | 0.94 | 0.35 | 1.00 | 0.27 | 1.00 | | |
| (-G143A) | 271.62 | 0.05 | 337.97 | 7.65 | 0.00 | 5.97* | 2000 + | | |
| | 0.54 | 0.10 | 0.58 | 0.04 | 0.10 | 0.04 | 0.66 | | |
| AKT1 | 0.17 | 0.14 | 0.02 | 0.45 | 0.10 | 1.00 | 0.06 | 0.24 | |
| (A13239T) | 1.48 | 1.70 | 1.04 | 2.64 | 1.52 | 0.00 | 1.14 | 0.57 | |
| | 0.00 | 0.02 | 0.00 | 0.01 | 0.01 | 0.07 | 0.00 | 0.01 | |
| AKT1 | 0.12 | 0.41 | 0.14 | 0.36 | 0.28 | 1.00 | 0.25 | 0.24 | 0.71 |
| (G18186A) | 1.03 | 0.35 | 2.37 | 9.00 | 0.53 | 0.00 | 4.02 | 5.58 | 0.14 |
| | 0.01 | 0.02 | 0.01 | 0.05 | 0.01 | 0.01 | 0.04 | 0.05 | 0.05 |
| ATK1 | 0.46 | 0.10 | 0.37 | 1.00 | 0.22 | 0.24 | 0.37 | 0.08 | 0.78 |
| (A20372G) | 2.90 | 1.51 | 2.22 | 2000 + | 2.26 | 0.57 | 2.27 | 0.82 | 32.9 |
| | 0.03 | 0.01 | 0.02 | 0.03 | 0.04 | 0.00 | 0.02 | 0.00 | 0.47 |
| AKT1 | 0.11 | 0.12 | 0.13 | 0.63 | 0.19 | 0.93 | 0.07 | 0.32 | 0.83 |
| (G20890A) | 1.55 | 1.34 | 1.73 | 7.85 | 1.66 | 0.05 | 1.37 | 0.58 | 20.1 |
| | 0.01 | 0.00 | 0.01 | 0.04 | 0.01 | 0.02 | 0.00 | 0.00 | 0.25 |

Linkage Disequilibrium among AKT1 SNPs: Caucasians

| | | | | | | | | | |
|---|---|---|---|---|---|---|---|---|---|
| AKT1 | 1.00 | | | | | | | | |
| (-C8678T) | 0.00 | | | | | | | | |
| | 0.05 | | | | | | | | |
| AKT1 | 0.99 | 1.00 | | | | | | | |
| (-C8541T) | 6520.73 | 0.00 | | | | | | | |
| | 0.94 | 0.05 | | | | | | | |
| AKT1 | 1.00 | 0.94 | 1.00 | | | | | | |
| (-C8371T) | 2000 + | 0.05 | 2000 + | | | | | | |
| | 0.38 | 0.02 | 0.36 | | | | | | |
| AKT1 | 1.00 | 0.98 | 1.00 | 1.00 | | | | | |
| (-C3349G) | 0.00 | 1507.41 | 0.00 | 0.00 | | | | | |
| | 0.03 | 0.61 | 0.03 | 0.01 | | | | | |
| AKT1 | 0.30 | 0.27 | 0.16 | 0.19 | 1.00 | | | | |
| (-G738T) | 2.81 | 4.67 | 1.69 | 2.92 | 0.00 | | | | |
| | 0.01 | 0.02 | 0.00 | 0.01 | 0.00 | | | | |
| AKT1 | 0.97 | 0.61 | 0.97 | 1.00 | 1.00 | 0.40 | | | |
| (-G171T) | 1012.05 | 0.27 | 1814.11 | 2000 + | 0.00 | 3.38 | | | |
| | 0.84 | 0.02 | 0.87 | 0.33 | 0.03 | 0.02 | | | |
| AKT1 | 0.74 | 0.46 | 0.87 | 0.28 | 1.00 | 0.25 | 0.97 | | |
| (-G143A) | 17.88 | 0.46 | 43.62 | 0.65 | 0.00 | 3.60 | 196.07 | | |
| | 0.23 | 0.00 | 0.31 | 0.00 | 0.01 | 0.02 | 0.36 | | |
| AKT1 | 0.32 | 0.55 | 0.30 | 0.64 | 0.57 | 0.22 | 0.35 | 0.14 | |
| (A13239T) | 3.64 | 5.75 | 3.39 | 8.52 | 5.72 | 1.87 | 4.38 | 0.77 | |
| | 0.08 | 0.08 | 0.07 | 0.12 | 0.05 | 0.00 | 0.11 | 0.00 | |
| AKT1 | 0.07 | 0.08 | 0.09 | 0.53 | 0.07 | 0.90 | 0.19 | 0.02 | 0.56 |
| (G18186A) | 0.88 | 0.90 | 0.86 | 0.38 | 1.48 | 0.08 | 0.70 | 0.97 | 0.29 |
| | 0.00 | 0.00 | 0.00 | 0.01 | 0.00 | 0.01 | 0.00 | 0.00 | 0.03 |
| ATK1 | 0.47 | 0.45 | 0.44 | 0.67 | 0.42 | 0.35 | 0.48 | 0.23 | 0.85 |
| (A20372G) | 4.87 | 3.38 | 4.48 | 7.35 | 2.96 | 2.33 | 5.47 | 1.90 | 49.7 |
| | 0.12 | 0.04 | 0.11 | 0.09 | 0.02 | 0.01 | 0.14 | 0.01 | 0.50 |
| AKT1 | 0.29 | 0.19 | 0.26 | 0.32 | 0.15 | 0.20 | 0.37 | 0.43 | 0.93 |
| (G20890A) | 2.98 | 3.06 | 2.60 | 6.09 | 2.31 | 2.85 | 3.68 | 0.49 | 77.9 |
| | 0.04 | 0.03 | 0.03 | 0.09 | 0.01 | 0.01 | 0.06 | 0.01 | 0.31 |

Linkage Disequilibrium among AKT1 SNPs: Hispanics

| | | | | | | | | | |
|---|---|---|---|---|---|---|---|---|---|
| AKT1 | 1.00 | | | | | | | | |
| (-C8678T) | 0.00 | | | | | | | | |
| | 0.08 | | | | | | | | |
| AKT1 | 0.62 | 0.85 | | | | | | | |
| (-C8541T) | 23.56 | 0.10 | | | | | | | |
| | 0.35 | 0.05 | | | | | | | |
| AKT1 | 0.72 | 1.00 | 0.75 | | | | | | |
| (-C8371T) | 24.31 | 0.00 | 29.12 | | | | | | |
| | 0.29 | 0.04 | 0.30 | | | | | | |
| AKT1 | 1.00 | 1.00 | 0.50 | 1.00 | | | | | |
| (-C3349G) | 0.00 | 2000 + | 0.39 | 0.00 | | | | | |
| | 0.06 | 0.72 | 0.01 | 0.03 | | | | | |
| AKT1 | 1.00 | 1.00 | 1.00 | 1.00 | 0.00 | | | | |
| (-G738T) | 0.00 | 2000 + | 0.00 | 0.00 | 1.00 | | | | |
| | 0.01 | 0.19 | 0.01 | 0.01 | 0.00 | | | | |
| AKT1 | 0.63 | 0.93 | 0.85 | 0.72 | 0.62 | 1.00 | | | |
| (-G171T) | 27.22 | 0.04 | 149.78 | 24.31 | 0.28 | 0.00 | | | |
| | 0.39 | 0.07 | 0.65 | 0.29 | 0.02 | 0.01 | | | |
| AKT1 | 0.47 | 0.41 | 0.75 | 1.00 | 0.01 | 1.00 | 1.00 | | |
| (-G143A) | 6.24 | 0.50 | 26.00 | 0.00 | 1.09 | 0.00 | 2000 + | | |
| | 0.07 | 0.00 | 0.25 | 0.02 | 0.00 | 0.00 | 0.33 | | |
| AKT1 | 0.10 | 0.24 | 0.35 | 0.68 | 0.59 | 1.00 | 0.10 | 1.00 | |
| (A13239T) | 1.34 | 1.99 | 2.67 | 7.44 | 5.68 | 0.00 | 1.34 | 0.00 | |
| | 0.00 | 0.02 | 0.04 | 0.09 | 0.09 | 0.04 | 0.00 | 0.07 | |
| AKT1 | 0.38 | 1.00 | 0.09 | 0.64 | 1.00 | 1.00 | 0.03 | 0.29 | 0.74 |
| (G18186A) | 5.88 | 0.00 | 1.73 | 0.28 | 0.00 | 0.00 | 1.16 | 3.69 | 0.12 |
| | 0.11 | 0.06 | 0.01 | 0.01 | 0.04 | 0.01 | 0.00 | 0.03 | 0.09 |
| ATK1 | 0.15 | 0.31 | 0.42 | 0.75 | 0.69 | 0.67 | 0.62 | 0.40 | 0.77 |
| (A20372G) | 1.48 | 2.24 | 3.05 | 8.83 | 5.09 | 0.19 | 5.92 | 2.51 | 29.5 |
| | 0.01 | 0.03 | 0.04 | 0.09 | 0.07 | 0.02 | 0.10 | 0.01 | 0.45 |
| AKT1 | 0.02 | 0.19 | 0.08 | 0.04 | 0.43 | 1.00 | 0.02 | 0.29 | 1.00 |
| (G20890A) | 0.96 | 2.60 | 1.49 | 1.23 | 6.54 | 0.00 | 0.96 | 0.64 | 200 |
| | 0.00 | 0.03 | 0.01 | 0.00 | 0.12 | 0.02 | 0.00 | 0.00 | 0.40 |

Linkage Disequilibrium among AKT1 SNPs: Others

| | | | | | | | | | |
|---|---|---|---|---|---|---|---|---|---|
| AKT1 | 1.00 | | | | | | | | |
| (-C8678T) | 0.00 | | | | | | | | |
| | 0.12 | | | | | | | | |
| AKT1 | 0.96 | 1.00 | | | | | | | |
| (-C8541T) | 4061.68 | 0.00 | | | | | | | |
| | 0.93 | 0.12 | | | | | | | |
| AKT1 | 1.00 | 1.00 | 0.42 | | | | | | |
| (-C8371T) | 2000 + | 0.00 | 0.50 | | | | | | |
| | 0.23 | 0.03 | 0.00 | | | | | | |
| AKT1 | 1.00 | 0.56 | 1.00 | 1.00 | | | | | |
| (-C3349G) | 0.00 | 8.22 | 0.00 | 0.00 | | | | | |
| | 0.06 | 0.16 | 0.06 | 0.01 | | | | | |
| AKT1 | 0.77 | 0.70 | 0.23 | 1.00 | 1.00 | | | | |
| (-G738T) | 0.16 | 12.43 | 0.69 | 0.00 | 0.00 | | | | |
| | 0.03 | 0.18 | 0.00 | 0.01 | 0.04 | | | | |
| AKT1 | 1.00 | 1.00 | 1.00 | 1.00 | 1.00 | 0.96 | | | |
| (-G171T) | 2000 + | 0.00 | 2000 + | 2000 + | 0.00 | 0.02 | | | |
| | 0.81 | 0.15 | 0.80 | 0.18 | 0.07 | 0.05 | | | |
| AKT1 | 1.00 | 1.00 | 1.00 | 1.00 | 1.00 | 0.92 | 1.00 | | |
| (-G143A) | 2000 + | 0.00 | 2000 + | 0.00 | 0.00 | 0.06 | 2000 + | | |
| | 0.49 | 0.05 | 0.49 | 0.01 | 0.03 | 0.02 | 0.39 | | |
| AKT1 | 1.00 | 0.30 | 1.00 | 1.00 | 0.65 | 0.37 | 1.00 | 1.00 | |
| (A13239T) | 2000 + | 0.40 | 2000 + | 2000 + | 0.15 | 2.40 | 2000 + | 2000 + | |
| | 0.24 | 0.04 | 0.23 | 0.05 | 0.10 | 0.02 | 0.29 | 0.12 | |
| AKT1 | 1.00 | 0.09 | 1.00 | 1.00 | 0.04 | 0.31 | 1.00 | 1.00 | 1.00 |
| (G18186A) | 0.00 | 0.88 | 0.00 | 0.00 | 1.23 | 0.63 | 0.00 | 0.00 | 0.00 |
| | 0.02 | 0.00 | 0.02 | 0.01 | 0.00 | 0.00 | 0.03 | 0.01 | 0.11 |
| ATK1 | 1.00 | 0.35 | 0.91 | 1.00 | 0.20 | 0.00 | 0.62 | 1.00 | 0.85 |
| (A20372G) | 2000 + | 0.34 | 34.55 | 2000 + | 0.61 | 1.00 | 6.12 | 2000 + | 100. |
| | 0.24 | 0.06 | 0.22 | 0.06 | 0.01 | 0.00 | 0.11 | 0.12 | 0.66 |
| AKT1 | 0.93 | 0.64 | 0.19 | 1.00 | 0.07 | 1.00 | 0.88 | 0.47 | 1.00 |
| (G20890A) | 180.20 | 0.24 | 2.42 | 2000 + | 1.50 | 0.00 | 65.24 | 13.58 | 200 |
| | 0.51 | 0.03 | 0.02 | 0.39 | 0.00 | 0.03 | 0.37 | 0.18 | 0.14 |

SUPPLEMENTAL TABLE 6
Rapidia volumetric measurements of randomly selected
genotypes.
Summary of significant results with AKT1 (-G171T), Volumetric associations, and variability
attributable to genotype effect
Volumetric and Cross-sectional area associations

| | | | | | | | r-squ |
|---|---|---|---|---|---|---|---|
| | | | | | | P-value for | with |
| | | | | | Significant differences | significant | genot |
| Measurement | Arm | Gender | F-test * | P-value* | (N; adjusted mean ± SEM) | differences | effect |
| Baseline | Exercised | Male | 3.91 | 0.027 | GG (N = 25; 93.54 ± 5.63) | * 0.025 | 0.2107 |
| marrow CSA | | | | | GT (N = 16; 78.39 ± 7.04) * | | |
| | | | | | TT (N = 10; 110.82 ± 9.39) * | | |
| Post-exercise | Exercised | Male | 3.48 | 0.039 | GG (N = 24; 89.24 ± 5.41) | * 0.035 | 0.1779 |
| marrow CSA | | | | | GT (N = 16; 80.74 ± 6.62) * | | |
| | | | | | TT (N = 10; 109.76 ± 8.84) * | | |
| Baseline bone + marrow | Exercised | Male | 10.61 | <0.001 | GG (N = 25; 304.64 ± 7.51) * | * 0.002 | 0.5372 |
| CSA | | | | | GT (N = 16; 285.66 ± 9.39) ** | ** <0.001 | |
| | | | | | TT (N = 10; 357.11 ± 12.52) *, ** | | |
| Post-exercise | Exercised | Male | 12.04 | <0.001 | GG (N = 24; 302.89 ± 7.83) * | * 0.001 | 0.5349 |
| bone + marrow | | | | | GT (N = 16; 283.42 ± 9.59) ** | ** <0.001 | |
| CSA | | | | | TT (N = 10; 360.97 ± 12.80) *, ** | | |
| Baseline fat | Exercised | Male | 5.83 | 0.006 | GG (N = 25; 1917.44 ± 142.29) * | * 0.008 | 0.5633 |
| CSA | | | | | GT (N = 16; 1964.45 ± 177.90) ** | ** 0.009 | |
| | | | | | TT (N = 10; 1042.40 ± 237.32) *, ** | | |
| Post-exercise | Exercised | Male | 5.43 | 0.008 | GG (N = 24; 1812.36 ± 145.56) * | * 0.020 | 0.5566 |
| fat CSA | | | | | GT (N = 16; 1984.45 ± 178.18) ** | ** 0.008 | |
| | | | | | TT (N = 10; 1018.90 ± 237.93) *, ** | | |
| Baseline | Exercised | Female | 3.39 | 0.038 | GG (N = 42; 159.76 ± 3.49) | * 0.040 | 0.3053 |
| cortical bone | | | | | GT (N = 33; 157.83 ± 3.89) * | | |
| CSA | | | | | TT (N = 11; 177.40 ± 6.70) * | | |
| Baseline | Exercised | Male | 7.97 | 0.001 | GG (N = 25; 211.11 ± 5.02) * | * 0.002 | 0.5387 |
| cortical bone | | | | | GT (N = 16; 207.26 ± 6.28) ** | ** 0.001 | |
| CSA | | | | | TT (N = 10; 246.29 ± 8.38) *, ** | | |
| Post-exercise | Exercised | Male | 7.96 | 0.001 | GG (N = 24; 213.66 ± 6.06) * | * 0.007 | 0.5050 |
| cortical bone | | | | | GT (N = 16; 202.68 ± 7.43) ** | ** 0.001 | |
| CSA | | | | | TT (N = 10; 251.21 ± 9.92) *, ** | | |
| Baseline | Non- | Male | 4.00 | 0.025 | GG (N = 25; 94.11 ± 4.98) | * 0.021 | 0.2771 |
| marrow CSA | exercised | | | | GT (N = 16; 84.04 ± 6.23) * | | |
| | | | | | TT (N = 10; 113.45 ± 8.31) * | | |
| Difference in | Non- | Male | 9.57 | <0.001 | GG (N = 24; −234.45 ± 5.88) * | * 0.004 | 0.5225 |
| marrow CSA | exercised | | | | GT (N = 16; −220.59 ± 7.20) ** | ** 0.001 | |
| | | | | | TT (N = 10; −272.68 ± 9.61) *, ** | | |
| Baseline bone + marrow | Non- | Male | 11.11 | <0.001 | GG (N = 25; 324.91 ± 7.85) * | * 0.002 | 0.5330 |
| CSA | exercised | | | | GT (N = 16; 303.56 ± 9.81) ** | ** <0.001 | |
| | | | | | TT (N = 10; 380.14 ± 13.09) *, ** | | |
| Post-exercise | Non- | Male | 10.55 | <0.001 | GG (N = 24; 323.74 ± 8.77) * | * 0.002 | 0.5035 |
| bone + marrow | exercised | | | | GT (N = 16; 303.61 ± 10.75) ** | ** <0.001 | |
| CSA | | | | | TT (N = 10; 384.84 ± 14.33) *, ** | | |
| Baseline fat | Non- | Male | 4.74 | 0.014 | GG (N = 25; 1961.87 ± 152.99) * | * 0.038 | 0.5475 |
| CSA | exercised | | | | GT (N = 16; 2144.84 ± 191.29) ** | ** 0.013 | |
| | | | | | TT (N = 10; 1191.45 ± 255.18) *, ** | | |

SUPPLEMENTAL TABLE 7
aqMan primer sets for SNPs tested.

| Gene | SNP | Forward Primer | Reverse Primer |
|---|---|---|---|
| AKT1 | -C12273T | GCCCAACTGGGAACATGAGA | CCGTGCCTCCTGCTGAG |
| AKT1 | -C8665T | GCCACTGGTGAAGGACGAA | AACAGGAGGTGGCTTCGG |
| AKT1 | -C8541T | CTGCCTTGGGCCAGACTT | CCAGGTCCCAGAAGAGTCAGA |
| AKT1 | -C8371T | GTCCGCGGTCCAGACA | TCCAGGAGAAGGATCGAAGTCT |
| AKT1 | -C3349G | ACCCCTTTCTCCTGGACACT | TGGAGGAACTTCTGGCTAGGAA |
| AKT1 | -G738A | CGGGAGGCCAGAAAGGT | TCTGTGGAATGGATCCCAACATG |
| AKT1 | -G171T | GGGCGCTGTGGTTTAGGA | CGCAAACGGGAGTCCAGAG |
| AKT1 | -G143A | GGGTTTCTCCCAGGAGGTTTT | GAAGACAGGACCAGGATGCA |
| AKT1 | A13239T | CACCAGGCCCCACGAT | CAGCCAGTGCTTGTTGCTT |
| AKT1 | G18186A | GAAGTCATCGTGGCCAAGGT | GCTGGGTGAGCTGCCA |
| AKT1 | A20372G | CTCAAGAAGGACCCCAAGCA | GGGCAGGTGCAGCCT |
| AKT1 | G20960A | CACGCTCGCCAGATTTCC | TGCAGCAGGCTCCTGAG |
| CNTF | -G6A | GGTGATGACAGAAGATGTGGTGTT | AGTCCAGGTTGATGTTCTTGTTCAG |
| IGF1 | -C1245T | GGATTTCAAGCAGAACTGTGTTTTCA | GGTGGAAATAACCTGGACCTTGAAT |
| ACTN3 | R577X | ACGATCAGTTCAAGGCAACACT | ACCCTGGATGCCCATGATG |
| IL6 | -G174C | GACGACCTAAGCTGCACTTTTC | GGGCTGATTGGAAACCTTATTAAGATTG |
| PPARg | P12A | GTTATGGGTGAAACTCTGGGAGATT | GCAGACAGTGTATCAGTGAAGGAAT |
| ACE* | I/D | CTGGAGACCACTCCCATCCTTTC | GATGTGGCCAATCACATTCGTC |

| | | WT allele probe | MT allele probe |
|---|---|---|---|
| Gene | SNP | (5′ VIC) | (5′ FAM) |
| AKT1 | -C12273T | TGGACCAGTGCCCTGT | TTTGGACCAGTTCCCTGT |
| AKT1 | -C8665T | CACTGTCCAAACAGG | CACTGTCCGAACAGG |
| AKT1 | -C8541T | CCTGGACCTGTCGTTG | CCTGGACCTATCGTTG |
| AKT1 | -C8371T | CAGGTTCTGCCCCAGGG | CAGGTTCTGTCCCAGGG |
| AKT1 | -C3349G | ACCTGCACTGTCCTGT | ACCTGCACTCTCCTGT |
| AKT1 | -G738A | CAGCTTAGACGCTCTC | CAGCTTAGATGCTCTC |
| AKT1 | -G171T | CAAGCCCAAAAAC | CAAGCACAAAAAC |
| AKT1 | -G143A | CTCTGGACTCCCGTTTG | TCTGGACTCCCATTTG |
| AKT1 | A13239T | CCCAGGACTTGGAG | CCCAGGACATGGAG |
| AKT1 | G18186A | CCGCACCCTCATCT | CCGCACCTTCATCT |
| AKT1 | A20372G | CCAGCTGCAGGCTA | TCCCAGCTACAGGCTA |
| AKT1 | G20960A | CACACTCGCCCTCAC | ACACACTCACCCTCAC |
| CNTF | -G6A | TTCCTGTATCCTCGGCCAG | TTCCTGTATCCTCAGCCA |
| IGF1 | -C1245T | CCTGAGAGTCATGTGGAAA | CTGAGAGTCATGCGGAA |
| ACTN3 | R577X | TCGCTCTCAGTCAGC | CGCTCTCGGTCAGC |
| IL6 | -G174C | CTTTAGCATGGCAAGAC | CTTTAGCATCGCAAGAC |
| PPARg | P12A | CTCCTATTGACCCAGAAAG | CTATTGACGCAGAAAG |
| ACE* | I/D | | |

| | | | | | | | |
|---|---|---|---|---|---|---|---|
| Post-exercise | Non- | Male | 5.09 | 0.010 | GG (N = 24; 1945.61 ± 167.47) * | * 0.026 | 0.4839 |
| fat CSA | exercised | | | | GT (N = 16; 2113.32 ± 205.16) ** | ** 0.011 | |
| | | | | | TT (N = 10; 1064.94 ± 273.94) *, ** | | |
| Baseline | Non- | Male | 8.29 | <0.001 | GG (N = 25; 230.80 ± 5.64) * | * 0.006 | 0.4884 |
| cortical bone | exercised | | | | GT (N = 16; 219.52 ± 7.05) ** | ** <0.001 | |
| CSA | | | | | TT (N = 10; 266.69 ± 9.41) *, ** | | |
| Post-exercise | Non- | Male | 9.60 | <0.001 | GG (N = 24; 231.92 ± 6.52) * | * 0.002 | 0.4809 |
| cortical bone | exercised | | | | GT (N = 16; 220.96 ± 7.99) ** | ** <0.001 | |
| CSA | | | | | TT (N = 10; 277.69 ± 10.66) *, ** | | |
| Baseline | Exercised | Male | 3.28 | 0.047 | GG (N = 25; 8676.9 ± 531.9) | * 0.045 | 0.1732 |
| marrow volume | | | | | GT (N = 16; 7336.1 ± 665.1) * | | |
| | | | | | TT (N = 10; 10132.8 ± 887.2) * | | |
| Baseline bone + marrow | Exercised | Male | 7.60 | 0.001 | GG (N = 25; 28212.9 ± 741.2) * | * 0.009 | 0.4372 |
| volume | | | | | GT (N = 16; 26736.2 ± 926.8) ** | ** 0.001 | |
| | | | | | TT (N = 10; 32683.3 ± 1236.3) *, ** | | |

What is claimed is:

1. A single nucleotide polymorphic polynucleotide (SNP) derived from the 12 kb region immediately upstream of the AKT1 gene in human males, selected from the group consisting of SEQ ID NO.s 1 through 8 or a complementary strand of SEQ ID Nos. 1 through 8, said polynucleotide exhibiting strong association with one or more of body composition and Body Mass Index (“BMI”) in said human males, said body composition consisting essentially of bone cortical volume, subcutaneous fat and volume, muscle volume and muscle strength.

2. The polynucleotide of claim 1, wherein said polynucleotide serves as an expression enhancer for the AKT1 gene in muscle.

3. The polynucleotide of claim 1, wherein the allele of said polynucleotide is designated as -G171T, and said polynucleotide has the sequence shown as SEQ ID No. 1.

4. The polynucleotide of claim 1, wherein the allele of said polynucleotide is designated as -C8541T, and said polynucleotide has the sequence shown as SEQ ID No. 2.

5. The polynucleotide of claim 1, wherein the allele of said polynucleotide is designated as -C12293T, and and said polynucleotide has the sequence shown as SEQ ID No. 3.

6. The polynucleotide of claim 1, wherein the allele of said polynucleotide is designated as -A8665G, and said polynucleotide has the sequence shown as SEQ ID No. 4.

7. The polynucleotide of claim 1, wherein the allele of said polynucleotide is designated -G738A, and said polynucleotide has the sequence shown as SEQ ID No.5.

8. The polynucleotide of claim 1, wherein said polynucleotide is designated -G143A, and said polynucleotide has the sequence shown as SEQ. ID No.6.

9. The polynucleotide of claim 1, wherein said polynucleotide is designated -C3349G, and said polynucleotide has the sequence shown as SEQ ID No. 7.

10. The polynucleotide of claim 1, wherein said polynucleotide is designated -G8371T, and said polynucleotide has the sequence shown as SEQ ID No. 8.

11. The polynucleotide of claim 1, wherein said polynucleotide is a member of a 4-allele haplotye (“Haplotype 2”), comprising SEQ ID Nos. 1, 2, 3 and 4, that is associated with increased baseline muscle strength.

12. The polynucleotide of claim 1, wherein said polynucleotide is a member of a 4-allele haplotye (“Haplotype 2”), comprising SEQ ID Nos. 1, 2, 3 and 4, that is associated with decreased subcutaneous fat.

13. The polynucleotide of claim 1, wherein said polynucleotide is a member of a 4-allele haplotype (“Haplotype 2”) comprising SEQ ID Nos. 1, 2, 3 and 4, that is associated with increased cortical volume.

14. The polynucleotide of claim 1, wherein said polynucleotide comprises SEQ ID NO. 5, that is associated with increased baseline muscle volume, and has no effect on obesity, bone volume or strength.

15. The polynucleotide of claim 1, wherein said polynucleotide comprises SEQ ID NO. 8 that is associated with increased subcutaneous fat and BMI.

16. A polynucleotide of claim 1 used in a method for predicting presymptomatically the likelihood that a human male will have a genetic propensity for a particular quality involving one or more of bone size, muscle volume and strength, volume of subcutaneous fat, and BMI, said method comprising the steps of obtaining a tissue sample from said human male, isolating genomic DNA from said tissue sample, then assaying said genomic DNA for one or more of the alleles of said polynucleotide.

17. The polynucleotide of claim 16, wherein said method comprises the steps of determining whether said human male presents with one or more of the alleles of the sequences of Haplotype 2, whereupon it can be predicted presymptomatically that said human male will have a genetic propensity for larger bones, larger and stronger muscles, a decreased amount of subsucateous adipose tissue, and a decreased BMI.

18. The polynucleotide of claim 17, wherein said allele constitutes a T for a G at position -171 in SEQ ID NO. 1.

19. The polynucleotide of claim 17, wherein said allele constitutes a T for a C at position -8541 in SEQ ID NO. 2.

20. The polynucleotide of claim 17, wherein said allele constitutes a A for a C at position -12293 in SEQ ID NO. 3.

21. The polynucleotide claim 17, wherein said allele constitutes a G for an A at position -8665 in SEQ ID NO. 4.

22. The polynucleotide of claim 16, wherein said method comprises the steps of determining whether said human male presents with the allele of SEQ ID NO. 5, whereupon it can be presymptomatically predicted that said human will have a propensity for larger baseline muscle volume, but no effect on subcutaneous fat, BMI, bone size, or muscular strength.

23. The polynucleotide of claim 22, wherein said allele constitutes an A for a G at position -738 in SEQ ID NO. 5.

24. The polynucleotide of claim 16, wherein said method comprises the steps of determining whether said human male presents with Haplotype 1, whereupon it can be predicted presymptomatically that said human will have an increased amount of subcutaneous fat. and an increased BMI.

25. The polynucleotide of claim 24, wherein said allele constitutes a T for a G at position -8371 in SEQ ID NO. 8.

26. The polynucleotide of claim 16, wherein said method comprises the steps of determining whether said human male presents with the T or a G allele at position -171 in Seq ID NO. 1 or an A for a T allele at position -143 in SEQ ID NO. 6.

27. The polynucleotide of claim 26, wherein said allele predicts an increase of AKT1 gene enhancer activity in muscle myotubules.

28. A method for predicting presymptomatically the likelihood that a human male will have a genetic propensity for a particular quality involving one or more of bone size, muscular development and strength, amount of subcutaneous fat, comprising the steps of obtaining a tissue sample from said human male, isolating genomic DNA from said tissue sample, then assaying said genomic DNA for one or more of the alleles of the SNPs of claim 1.

29. A method of claim 28, comprising the steps of determining whether said human male presents with one or more of the alleles of the sequences of Haplotype 2, whereupon it can be predicted presymptomatically that said human male will have a genetic propensity for larger bones, larger and stronger muscles, a decreased amount of subcutaneous adipose tissue, and a decreased BMI.

30. The method of claim 29, wherein said allele constitutes a T for a G at position -171 in SEQ ID NO. 1.

31. The method of claim 29 herein said allele constitutes a T for a C at position -8541 in SEQ ID NO. 2.

32. The method of claim 29, wherein said allele constitutes a A for a C at position -12,293 in SEQ ID NO. 3.

33. The method of claim 29, wherein said allele constitutes a G for an A at position -8665 in SEQ ID NO. 4.

34. The method of claim 28, comprising the steps of determining whether said human male presents with the allele of SEQ ID NO. 5, whereupon it can be presymptomatically predicted that said human will have a propensity for larger baseline muscle volume, but no effect on subcutaneous fat, BMI, bone size, or muscular strength.

35. The method of claim 34, wherein said allele constitutes an A for a G at position -738 in SEQ ID NO. 5.

36. The method of claim 28, comprising the steps of determining whether said human male presents with Haplotype 1, whereupon it can be predicted presymptomatically that said human will have an increased volume of subcutaneous fat and an increased BMI.

37. The method of claim 36, wherein said allele constitutes a T for a G at position -8371 in SEQ ID NO. 8.

38. The method of claim 28, comprising the steps of determining whether said human male presents with the T or a G allele at position -171 in Seq ID NO. 1 or an A for a T allele at position -143 in SEQ ID NO. 6.

39. The method of claim 38, wherein said alleles predict an increase of AKT1 gene enhancer activity in muscle myotubes.

40. A diagnostic reagent comprising one or more SNPs of claim 1 in a form suitable for use in detecting the related allele in a sample of genomic DNA of a human male.

41. A commercial kit containing one or more of the SNPs of claim 1 in a physical form appropriate for use in presymptomatic diagnostic methodology related to evaluating clinical interventions for improving body composition and muscle strength and avoiding, increased BMI, and enhancing AKT1 gene expression in myotubules.


SEQUENCE LISTING (8 sequences)

SEQ ID NO: 1 - DNA, 60 bp, Homo sapiens
  allele (16): G 171 T
cccaggaggtttttgkgcttgcctggagggcccaggaggtttttggcttgcgctggaggg

SEQ ID NO: 2 - DNA, 62 bp, Homo sapiens
  allele (17): C 8541 T
gccagaacttcaacgayaggtccagggcctgggccagacttcaaacgaggtccagggcctgg

SEQ ID NO: 3 - DNA, 61 bp, Homo sapiens
  allele (16): C 12273 A
ccctgcaggacagggmactggtccaaactcaccctgcaggacagggactggtccaaactca

SEQ ID NO: 4 - DNA, 61 bp, Homo sapiens
  allele (16): G 8665 A
tgctctcctcctgttrggacagtgagtcccgtgctctcctcctgttggacagtgagtcccg

SEQ ID NO: 5 - DNA, 60 bp, Homo sapiens
  allele (16): G 738 A
gcctggggggagagcrtctaagctggctgtgcctggggggagagcctctagctggctgtg

SEQ ID NO: 6 - DNA, 61 bp, Homo sapiens
  allele (16): G 143 A
gggctctggactcccrtttgcgccagtggccgggctctggactccctttgcgccagtggcc

SEQ ID NO: 7 - DNA, 61 bp, Homo sapiens
  allele (16): C 3349 G
tggacactcacaggasagtgcaggtgactgctggacactcacaggaagtgcaggtgactgc

SEQ ID NO: 8 - DNA, 61 bp, Homo sapiens
  allele (16): C 8371 T
gctgagcaggttctgycccaggggcagggttgctgagcaggttctgcccaggggcagggtt